(19) United States
(12) Patent Application Publication
(10) Pub. No.: US 2026/0250641 A1
(43) Pub. Date: Aug. 27, 2026

(54) COMPOSITIONS AND USES THEREOF FOR STABILIZATION OF BLOOD SAMPLE COMPONENTS

(71) Applicant: STRECK LLC, La Vista, NE (US)

(72) Inventors: Jing Li Oupicky, La Vista, NE (US); Nicholas Michael George, Omaha, NE (US); Sean Salamifar, Papillion, NE (US); Nursen Binbuga, Omaha, NE (US)

(21) Appl. No.: 19/164,483

(22) PCT Filed: Mar. 14, 2024
(86) PCT No.: PCT/US2024/019914

§ 371 Date: Sep. 11, 2025

Related U.S. Application Data
(60) Provisional application No. 63/490,225, filed on Mar. 14, 2023.

(51) Int. Cl. C12N 5/00

(52) U.S. Cl. CPC C12N 5/526

(57) ABSTRACT

Provided herein are compositions comprising (1) a citrate-based anticoagulant (AC), an aldehyde releaser (AR), a calcium channel blocker, and a sodium channel blocker and (2) a citrate-based anticoagulant (AC), an aldehyde releaser (AR), and a sodium channel blocker, and uses of such compositions for stabilizing components in a blood sample.

### BACKGROUND

Highly anticipated advancement in biotechnology and instrumentation now allows for simultaneous detection of multiple biomarkers and multi-omics profiling. This approach is powering discovery for new biomarkers and druggable targets across multiple dimensions of biological alterations by providing panoramic landscapes and integrated analysis of any disease. It is accelerating the cancer research field towards the development of multianalyte-based in vitro diagnostic tests for early detection and/or monitoring of cancer. These multianalyte liquid biopsy tests are often based on detection of cancer-related proteins and/or tumor-specific genomic and transcriptomic changes (circulating cell-free nucleic acids) in plasma. As these tests enable more comprehensive analysis from a single blood sample (e.g., venous and/or capillary blood samples) and often require shipping and handling from blood collection sites to a centralized testing site, sample integrity becomes extremely critical. While innovations in automation and instrumentation have significantly reduced analytical errors over the past decade, pre-analytical errors and variables introduced before sample testing remain the top concerns. More importantly, many biomarkers of interest in a blood specimen are either sensitive to degradative enzymes or prone to contamination of degradation products released from blood cells.

During storage, especially ambient temperature storage, blood cells go through apoptosis due to energy and/or environmental stress. White blood cells (WBC) contain genomic DNA, the fragments of which will be released from apoptotic WBC into plasma when WBC lyse. This will result in elevated level of total cell-free DNA (cfDNA) and masking of tumor-derived circulating cfDNA. Lysed blood cells, such as apoptotic reticulocytes and WBC can also release cellular RNA fragments proteins and peptides into plasma, hindering the quantification of tumor-derived cfRNA fragments. Besides blood cells, activated platelets can also greatly alter plasma protein profile. The α-granules in platelets contain various kinds of proteins, including adhesive glycoproteins, coagulation factors, mitogenic factors, angiogenic factors, fibrinolytic inhibitors, immunoglobulins, chemokines, cytokine, and peptides. The activation of platelets triggers the release of α-granules contents into plasma, leading to altered population and concentration of plasma proteins. Hence, preventing platelet activation is crucial to maintain the draw time concentration and population of plasma protein, cytokine, and peptides.

Platelet activation can be initiated through various pathways. In the case of vascular injury, platelet activation is triggered by the binding of lamin, collagen, fibrinogen, and VWF to the adhesion receptors on platelet surface. EDTA, the commonly used anticoagulant in many stabilization blood collection tubes, also induces in vitro platelet activation, through reactions between EDTA-dependent antibodies and platelet glycoprotein IIb/Illa. Consequently, an ideal collection device for plasma protein assays will contain a none or low EDTA anticoagulant and a preservative formulation that can minimize the lysis of blood cells and prevent platelet from activation during an extended period of storage at room temperature.

A few commercial options for plasma protein stabilization in whole blood exist, including BD™ P100, BD™ P700, and BD™ P800 Blood Collection Tubes. These products contain K₂EDTA as anticoagulant and a protease inhibitors cocktail. Protease inhibitors can prevent plasma protein degradation but have a limited role in blocking cellular proteins from being released into plasma by apoptotic blood cells and activated platelets. As described in U.S. Ser. No. 14/003,033 and U.S. Ser. No. 13/757,080, usage of these tubes may be limited to a group of biomarkers. For instance, BD™ P700 was recommended by the manufacturer for the quantitation of glucagon-like peptide 1. BDT P800 is only intended to be used for plasma metabolic markers, such as glucagon, glucagon-like-peptide 1, and oxyntomodulin. Furthermore, these products are unable to stabilize cell-free nucleic acids during storage.

Magbio's Blood STASIS™-Omics Blood Collection Tube is the lone blood collection device on the market that claims to maintain the integrity of genomic DNA, mRNA, proteins, and metabolites. Nevertheless, the product does not claim to stabilize circulating cell-free nucleic acids.

Therefore, multi-omic blood tests that target both circulating nucleic acids and plasma proteins will require a different kind formulation to reduce pre-analytical variables. This kind of formulation will maintain the concentration and population of both cell-free nucleic acids and plasma proteins during storage and transportation. Often times, storing samples, e.g., blood samples, at refrigerated temperatures soon after collection is not possible or inconvenient. Thus, there is a need for improved compositions for the collection and ambient temperature storage of a blood sample and methods of preparing blood samples for multi-omics analysis.

### SUMMARY

In one aspect, the disclosure provides compositions comprising (1) a citrate-based anticoagulant (AC), an aldehyde releaser (AR), a calcium channel blocker, and a sodium channel blocker and (2) a citrate-based anticoagulant (AC), an aldehyde releaser (AR), and a sodium channel blocker.

Also provided are methods of stabilizing a blood sample comprising contacting a blood sample comprising with a composition described herein, wherein the composition stabilizes proteins, cytokines, polypeptides, DNA, RNA, rare blood cells, and/or extracellular vesicles within the blood sample.

### BRIEF DESCRIPTION OF THE DRAWINGS

FIG. 1. Perspective formulations with a higher concentration of mexiletine behaved better than those with lower or no mexiletine. Compared to Streck MH Formulation, blood samples treated with STRECK MO Formulation had much less increase in % activated platelet and plasma level of TGF-β1 during 3-day room temperature storage, indicating its superior ability in minimizing platelet activation.

FIG. 2. Compared to blood samples collected in EDTA tubes, Streck MO formulation maintained the draw time levels of plasma proteins of interest for up to 5 days of room temperature storage in whole blood. In plasma samples collected from EDTA tubes, protein biomarkers such as IL-8/CXCL8, TGF-β1, CCL5/RANTES, MMP-9 exhibited marked increase during whole blood storage at ambient temperature due to granule content release upon in vitro platelet activation and/or white blood cell degradation.

FIG. 3. Compared with whole blood samples stored at EDTA tubes, Streck MO formulation minimizes in vitro hemolysis post-draw (N=15). In plasma samples collected from EDTA tubes, significant hemolysis was observed in day 3 and day 5 samples, demonstrated by spectrometry analysis.

FIG. 4. Venous whole blood samples collected with EDTA (far left), ACD-A (middle), and Streck MO formulation (far right), post room-temperature storage, were labeled with CD62P and CD61 antibodies, followed by flow cytometry analysis. Streck MO Formulation is compatible with downstream flow cytometry analysis. In addition, Streck MO formulation minimized platelets activation for up to 7 days during ambient storage.

FIG. 5. Streck MO formulation shows stabilized cell free DNA (cfDNA) up to 7 days in drawn whole blood stored at ambient temperature.

FIG. 6. Streck MO formulation stabilized cell free RNA (cfRNA) up to 7 days in drawn whole blood stored at ambient temperature.

FIG. 7. Streck MO Formulation prevented platelet activation in venous blood samples during ambient storage (up to 3 days). Adding Streck MO formulation to capillary blood collected in the EDTA tube did not reverse platelet activation. Streck MO formulation reduced hemolysis in both venous blood and capillary blood.

FIG. 8. Streck MO Formulation minimized the in vitro hemolysis for up to 3 days in both venous blood and capillary blood. Streck MO Formulation prevented granule release from activated platelets in both venous blood samples and capillary blood for up to 3 days during room temperature storage.

FIG. 9. Streck MO Formulation (third set of data from the left) shows superior performance compared to EDTA tubes (first set of data from the left), ACD-A tubes (second set of data from the left), and BD P800 tubes (fourth set of data from the left) in maintaining draw time levels of plasma proteins of interest (RANTES, GranB, BDFN, MMP-9) in whole blood storage at ambient temperature.

FIG. 10. Compared to blood samples stored with EDTA, blood stored in tubes that were spray-coated with Streck formulations (MH, MH1.5 and M2) had smaller protein concentration changes during ambient storage.

FIG. 11. Ambient storage of EDTA blood led to genomic DNA (β-Actin) contamination, which will interfere with the downstream analysis cfDNA. Spray coated Streck formulations (MH and MH1.5) prevented genomic DNA contamination during the storage of blood. Spray-coated Streck M2 formulation also greatly reduced storage related genomic DNA contamination.

### DETAILED DESCRIPTION

Described herein are compositions comprising components which function together to (i) preserve cell morphology, stabilize cell structure, and/or prevent or reduce cell or cell fragment degradation, thereby reducing or preventing cell lysis and subsequent release of cellular proteins, and (ii) prevent or reduce protein degradation through the actions of deleterious proteolytic enzymes (e.g., thrombin, plasmin); and (iii) release of additional proteins from cells. Also provided are methods of stabilizing a blood sample comprising contacting a blood sample comprising with a composition described herein, wherein the composition stabilizes proteins, cytokines, blood cells (including PBMC and rare blood cells), DNA, RNA and/or extracellular vesicles including EV cargoes and EV surface markers) within the blood sample.

The composition described herein comprises (1) a citrate-based anticoagulant (AC), an aldehyde releaser (AR), a calcium channel blocker, and a sodium channel blocker and (2) a citrate-based anticoagulant (AC), an aldehyde releaser (AR), and a sodium channel blocker. The term anticoagulant (AC) refers to an agent that inhibits the coagulation of blood. In exemplary aspects, the AC is ethylene diamine tetra acetic acid (EDTA) or a salt thereof, ethylene glycol tetra acetic acid (EGTA) or a salt thereof, hirudin, heparin, citric acid, a salt of citric acid, oxalic acid, a salt of oxalic acid, acid citrate dextrose (ACD; also known as anticoagulant citrate dextrose), citrate-theophylline-adenosine-dipuridamole (CTAD), citrate-pyridoxalphosphate-tris, heparin-1,3-hydroxy-ethyl-theophylline, polyanethol sulfonate, sodium polyanethol sulfonate, sodium fluoride, sodium heparin, thrombin and PPACK (D-phenylalanyl-L-prolyl-L-arginine chloromethyl ketone), or a combination thereof. In exemplary aspects, the anticoagulant is EDTA. Optionally, EDTA is the only AC present in the protective agent.

In some embodiments, the anticoagulant is a citrate-based anticoagulant (AC). Exemplary citrate-based ACs include, acid citrate dextrose (ACD, i.e., citric acid, trisodium citrate and dextrose), citrate, citrate-theophylline-adenosine-dipyridamole (CTAD), citrate-pyridoxalphosphate-tris, citrate-dextrose-phosphate-adenine (CDPA), citrate-phosphate-dextrose-adenine (CPDA), or a combination thereof.

In exemplary aspects, the citrate-based AC is present in the composition in an amount of about 10 g/L to about 500 g/L, about 10 g/L to about 450 g/L, about 10 g/L to about 400 g/L, about 10 g/L to about 350 g/L, about 10 g/L to about 300 g/L, about 10 g/L to about 250 g/L, about 10 g/L to about 200 g/L, about 10 g/L to about 150 g/L, about 10 g/L to about 100 g/L, about 10 g/L to about 75 g/L, about 10 g/L to about 50 g/L, about 50 g/L to about 500 g/L, about 75 g/L to about 500 g/L, about 100 g/L to about 500 g/L, about 150 g/L to about 500 g/L, about 200 g/L to about 500 g/L, about 250 g/L to about 500 g/L, about 300 g/L to about 500 g/L, about 350 g/L to about 500 g/L, about 400 g/L to about 500 g/L, about 450 g/L to about 500 g/L, about 20 g/L to about 400 g/L, about 30 g/L to about 300 g/L, about 40 g/L to about 250 g/L, or about 50 g/L to about 200 g/L. In certain instances, the anticoagulant is present in the composition in an amount of about 50 g/L to about 200 g/L, about 50 g/L to about 150 g/L or about 60 g/L to about 100 g/L. In some aspects, about 50 g/L, about 60 g/L, about 70 g/L, about 80 g/L, about 90 g/L, about 100 g/L, about 150 g/L or about 200 g/L anticoagulant is present in the composition.

In some embodiments, the AC is acid citrate dextrose (ACD, i.e., citric acid, trisodium citrate and dextrose). In some embodiments, the composition comprises citric acid at a concentration of about 10 g/L to about 50 g/L citric acid (or about 10 g/L to about 15 g/L, or 10 g/L to about 40 g/L, or about 15 g/L to about 30 g/L, or about 20 g/L to about 50 g/L). In some embodiments, the composition comprises citric acid at a concentration of 10 g/L, about 11 g/L, about 12 g/L, about 13 g/L, about 14 g/L, about 15 g/L, about 16 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 41 g/L, about 42 g/L, about 43 g/L, about 44 g/L, about 45 g/L, about 46 g/L, about 47 g/L, about 48 g/L, about 49 g/L or about 50 g/L. In some embodiments, the composition comprises trisodium citrate at a concentration of about 10 g/L to about 200 g/L (or about 20 g/L to about 50 g/L, or about 50 g/L to 75 g/L, or about 50 g/L to about 150 g/L, or about 60 g/L to about 100 g/L, or about 75 g/L to about 200 g/L). In some embodiments, the composition comprises trisodium citrate at a concentration of about 10 g/L, about 20 g/L, about 30 g/L, about 40 g/L, about 50 g/L, about 60 g/L, about 70 g/L, about 80 g/L, about 90 g/L, about 100 g/L, about 150 g/L, about 200 g/L. In some embodiments, the composition comprises dextrose at a concentration of about 30 g/L to about 180 g/L (or about 30 g/L to about 75 g/L, or about 50 g/L to about 100 g/L, or about 75 g/L to about 10 g/L, or about 78 g/L to about 180 g/L, or about 65 g/L to about 160 g/L). In some embodiments, the composition comprises dextrose at a concentration of about 30 g/L, about 40 g/L, 50 g/L, about 55 g/L, about 60 g/L, about 65 g/L, about 70 g/L, about 71 g/L, about 72 g/L, about 73 g/L, about 74 g/L, about 75 g/L, about 80 g/L, about 85 g/L, about 90 g/L, about 95 g/L, about 100 g/L, about 105 g/L, about 110 g/L, about 115, about 120 g/L, about 125 g/L, about 130 g/L, about 135 g/L, about 140 g/L, about 145 g/L, about 150 g/L, about 155 g/L, about 160 g/L, about 165 g/L, about 170 g/L, about 175 g/L, or about 180 g/L.

The composition comprises an aldehyde releaser (AR) (i.e., an agent that reacts to form an aldehyde product, e.g., a formaldehyde product). In exemplary aspects, the AR reacts to provide a slow release of the aldehyde product over time and without being bound to a particular theory, the slow release of the aldehyde product by the AR imparts stability to the blood sample, e.g., the cellular components of the blood sample. In exemplary embodiments, the aldehyde releaser is diazolidinyl urea, imidazolidinyl urea, 1,3,5-tris(hydroxyethyl)-s-triazine, oxazolidine, 1,3-bis(hydroxymethyl)-5,5-dimethylimidazolidine-2,4-dione, quaternium-15, DMDM hydantoin, 2-bromo-2-nitropropane-1,3-diol, 5-bromo-5-nitro-1,3-dioxane, tris(hydroxymethyl) nitromethane, glyoxal, acrolein, furfural, hydroxymethylglycinate, polyquaternium, or a combination thereof. In exemplary aspects, the aldehyde releaser is imidazolidinyl urea. In some embodiments, imidazolidinyl urea is the only AR in the composition.

Additional exemplary aldehyde releasers are provided below in Table A.

TABLE A

| Chemical Name | CAS Registry # | Chemical Structure |
|---|---|---|
| Benzylhemiformal (Methanol, (phenylmethoxy-)) | 14548-60-8 | 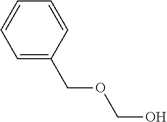 |
| 5-bromo-5-nitro- 1,3-dioxane | 30007-47-7 | 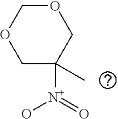 |
| Diazolidinyl urea (Germall II) | 78491-02-8 | 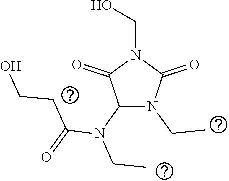 [SMILES: CCN(C(=O)CCO)C1C(=O)N(CO)C(=O)N1CC] |
| 1,3-dimethylol-5,5- dimethylhydantoin (or DMDM hydantion) | 6440-58-0 | 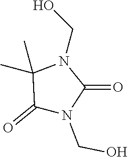 |
| Imidazolidinyl urea (Germal 115) | 39236-46-9 | 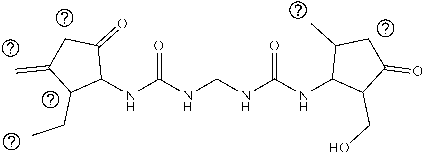 [SMILES: C=C1CC(=O)C(NC(=O)NCNC(=O)NC2C(C)CC(=O)C2CO)C1CC] |
| Sodium hydroxymethylglycinate | 274-357-8 | 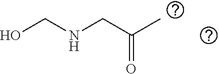 |
| Polynoxylin (Polyoxymethylene urea) | 9011-05-6 | 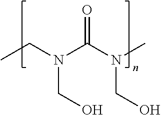 |
| Formaldehyde | 50-00-0 | ═O |
| ethylenedioxy) dimethanol | 3586-55-8 | 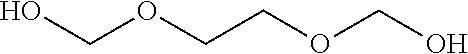 |
| Tetramethylol- acetylenediurea | 5395-50-6 | 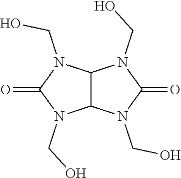 [SMILES: O=C1N(CO)C2C(N1CO)N(CO)C(=O)N2CO] |
| Methenamine (Urotropin) | 100-97-0 | 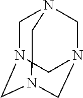 |
| Glutaraldehyde | 111-30-8 | 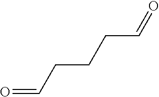 |
| N-methylol- choracetamide | 2832-19-1 | 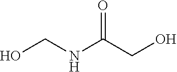 |
| Chlormethylacylamino methanol | 16473-35-1 | 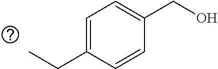 |
| Hexamidine | 3811-75-4 | 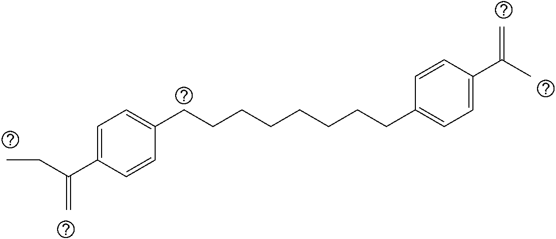 [SMILES: C=C(C)c1ccc(CCCCCCCCc2ccc(C(=C)CC)cc2)cc1] |
| 4-[2-(Morpholin- 4-ylmethyl)- 2-nitro- butyl]morpholine | 37304-88-4 | 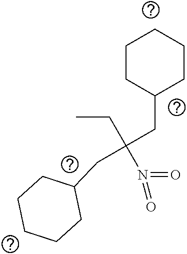 [SMILES: CCC(CC1CCCCC1)(CC1CCCCC1)[N+](=O)[O-]] |
| 1,3-Dimethyl-4,5- dihydroxyethyleneurea | 3923-79-3 | 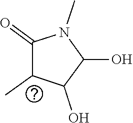 |
| Dimethylolethyleneurea | 136-84-5 | 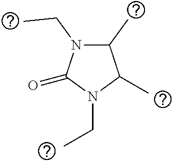 |
| Dimethyloldihydroxy- ethyleneurea | 1854-26-8 | 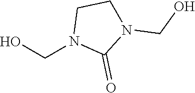 |
| Dimethylolpropyleneurea | 3270-74-4 | 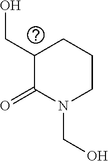 [SMILES: O=C1C(CO)CCCN1CO] |
| Dimethylol urea | 140-95-4 | 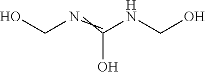 |
| Ethylene urea | 120-93-4 | 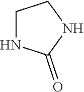 |
| (Z)-3-(Bis(2- hydroxyethyl) amino)-2-(2- hydroxyethyl- (hydroxymethyl)amino) prop-2-en-1-ol | 77044-78-1 | 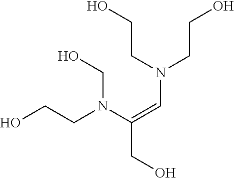 [SMILES: OCCN(/C=C(/CO)N(CO)CCO)CCO] |
| 1,3,5-Triethyl-1,3,5- triazinane | 7779-27-3 | 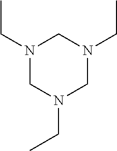 |
| Glyoxalmonourein | 3720-97-6 | 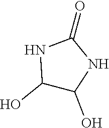 |
| MDM hydantoin | 116-25-6 | 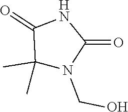 |
| 4,4′-Methylene- dimorpholine | 5625-90-1 | 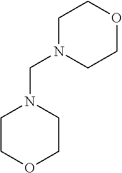 [SMILES: C1CN(CN2CCOCC2)CCO1] |
| Methylol urea ((HYDROXYMETHYL) UREA) | 10082-4 | 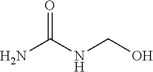 |
| 3,4,4- trimethyloxazolidine | 75673-43-7 | 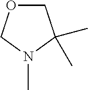 |
| 2,2′,2″-(1,3,5- Triazine-2,4,6-triyl) tri(ethan-1-ol) | 20922-84-3 | 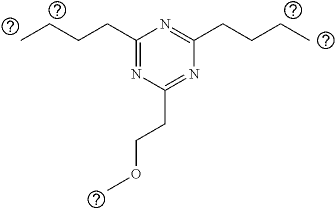 [SMILES: CCCCc1nc(CCCC)nc(CCOC)n1] |
| 4,4-DIMETHYL- OXAZOLIDINE | 51200-87-4 | 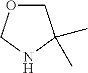 |
| 4,4′-(2-Ethyl-2- nitrotrimethylene) dimorpholine | 1854-23-5 | 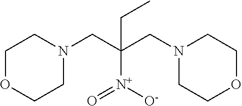 [SMILES: CCC(CN1CCOCC1)(CN1CCOCC1)[N+](=O)[O-]] |
| 4-(2-Nitrobutyl) morpholine | 2224-44-4 | 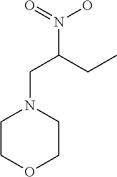 |
| Polyoxymethylene melamine (Melamine-urea- formaldehyde) | 25036-13-9 | 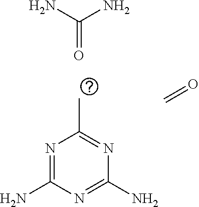 |
| Phenylmethoxy- methoxymethylbenzene | 2749-70-4 | 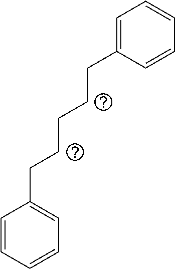 [SMILES: c1ccc(CCCCCc2ccccc2)cc1] |
| Propyleneglycol hemiformal (sodium hydroxymethylglycinate) | 70161-44-3 | 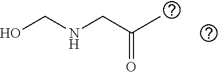 |
| Hydantoin | 461-72-3 | 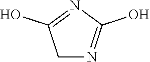 |
| 3-(Hydroxymethyl)-5,5- dimethylimidazolidine- 2,4- dione | 16228-00-5 | 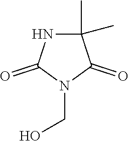 |
| Methylal | 109-87-5 | 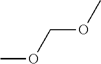 |
| N-Methylethanolamine | 109-83-1 | 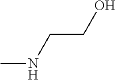 |
| Oxazolidine (3,3′- Methylenebis(5- methyloxazolidine)) | 66204-44-2 | 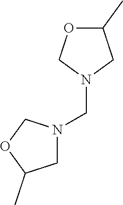 |
| 5-hydroxymethoxy- methyl-1-laza-3,7- dioxabicyclo[3.3.0] octane | 59720-42-2 | 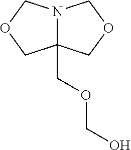 [SMILES: OCOCC12COCN1COC2] |
| Dithio-bis(male- imidoethane) (DTME) | 71865-37-7 | 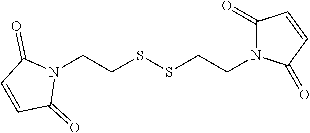 [SMILES: O=C1C=CC(=O)N1CCSSCCN1C(=O)C=CC1=O] |
| Ortho-phthalaldehyde (OPA) | 643-79-8 | 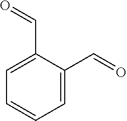 |
| p-phthalaldehyde (Terephthalaldehyde) | 623-27-8 | 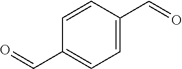 |
| Urea, polymer with formaldehyde, isobutylated | 68002-18-6 | 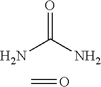 |
| Polyoxymethylene melamine | 9003-08-1 | 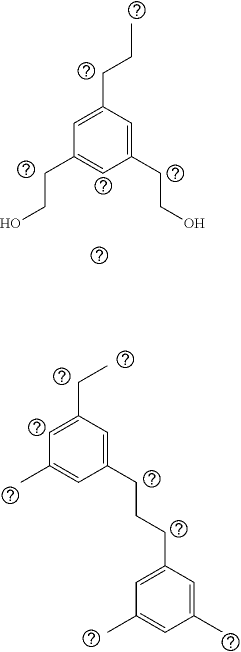 |
| Tris (hydroxymethyl)- nitromethane | 126-11-4 | 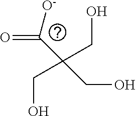 |
| Dimethylhydantoin formaldehyde resin (DMHF) | 26811-08-5 or 9065-13-8 | 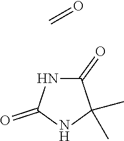 |
| 2-Imidazolidinone (4,5-hydroxy-1,3- bis(hydroxymethyl)-, methylated) | 68411-81-4 | 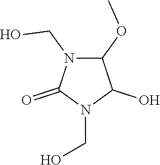 |
| 7a-Ethyldihydro- 1H,3H,5H- oxazolo(3,4-c)oxazole | 7747-35-5 | 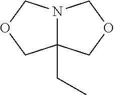 |
| Bioban CS 1135 | 81099-36-7 | 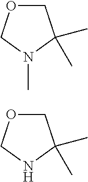 |
| 2,4-Imidazolidinedione, 3-(hydroxymethyl)-5,5- dimethyl | 16228-00-5 | 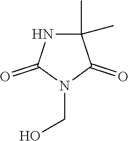 |
| Bis(hydroxymethyl)urea | 25155-29-7 | 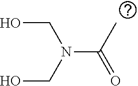 |
| 2- [(HYDROXYMETHYL) AMINO]ETHANOL | 34375-28-5 | 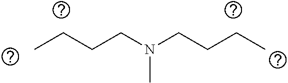 |
| Polyoxymethylene | 9002-81-7 | 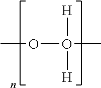 |
| Timonacic (1,3- thiazolidane- 4-carboxylic acid) | 60731-25-1 | 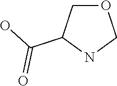 |
| Tris(N-hydroxyethyl) hexadrotriazine | 25254-50-6 | 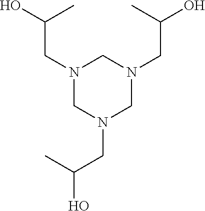 [SMILES: CC(O)CN1CN(CC(C)O)CN(CC(C)O)C1] |
| Poly(p- toluenesulfonamide- co-formaldehyde)- PTSAF | 25035-71-6 | 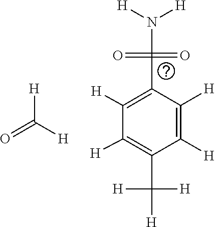 |

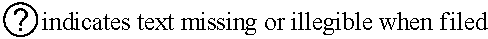

In some embodiments, the aldehyde releaser is present in the composition in an amount of about 100 g/L to about 1000 g/L, about 200 g/L to about 1000 g/L, or about 300 g/l to about 1000 g/l, about 500 g/L to about 1000 g/L, about 600 g/L to about 1000 g/L, about 700 g/L to about 1000 g/L, about 800 g/L to about 1000 g/L, about 900 g/L to about 1000 g/L, about 100 g/L to about 900 g/L, about 100 g/L to about 800 g/L, about 100 g/L to about 700 g/L, about 100 g/L to about 600 g/L, about 100 g/L to about 500 g/L, about 100 g/L to about 400 g/L, about 100 g/L to about 300 g/L, about 100 g/L to about 200 g/L, or about 140 g/L to about 160 g/L (e.g., about 150 g/L, about 300 g/L, about 400 g/L, about 500 g/L, about 600 g/L, or about 700 g/L). In exemplary aspects, the aldehyde releaser is present in the composition in an amount of about 100 g/L, about 150 g/L, about 200 g/L, about 250 g/L, about 300 g/L, about 350 g/L, about 400 g/L, about 450 g/L, about 500 g/L, about 550 g/L, about 600 g/L, about 700 g/L, about 800 g/L, about 900 g/L, or about 1000 g/L, ±10% g/L. In some embodiments, the composition comprises imidazolidinyl urea at a concentration of about 100 g/L to about 400 g/L, or about 300 g/L to about 700 g/L or about 140 g/L to about 160 g/L. In some embodiments, the composition comprises imidazolidinyl urea at a concentration of 150 g/L.

In some embodiments, the composition comprises the AR and the AC at an AC to AR ratio of about 4:1, or about 4:1 to about 1:6, or about 4:1 to about 1:2, or about 2:1, or about 1:1 to about 1:2, or about 1:2 to about 1:1, or about 1:2 to about 1:6, or about 2:1 to about 1:6. In some aspects, the composition comprises the AR and the AC at an AC to AR ratio of about 1:3 to about 1:5. In some embodiments, the composition comprises the AR and the AC at an AC to AR ratio of about 1:4. In some embodiments, the composition comprises the AR and the AC at an AC to AR ratio of about 1.026:1. In some embodiments, the composition comprises the AR and the AC at an AC to AR ratio of about 2.05:1. In some embodiments, the composition comprises the AR and the AC at an AC to AR ratio of about 1.54:1. In some embodiments, the composition comprises the AR and the AC at an AC to AR ratio of about 1:1.2.

In some embodiments, the composition further comprises a calcium channel blocker in an amount ranging from 1 g/L to about 20 g/L (or from about 2 g/L to about 20 g/L, or from or about 1 g/L to about 5 g/L, about 2 g/L to about 5 g/L, or about 1 g/L to about 10 g/L, or from about 2 g/L to about 10 g/L, or from about 5 g/L to about 20 g/L, or from about 5 g/L to about 10 g/L, or from about 4 g/L to about 10 g/L). In some embodiments, the composition comprises a calcium channel blocker in an amount of about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 16 g/L, about 17 g/L, about 18 g/L, about 19 g/L or about 20 g/L Exemplary calcium channel blockers include, but are not limited to, amlodipine, felodipine, isradipine, nicardipine, nisoldipine, verapamil, diltiazem, and nifedipine. In some embodiments, the calcium channel blocker is verapamil.

In some embodiments, the composition comprises verapamil in an amount ranging from about 1 g/L to about 20 g/L or from (or from about 2 g/L to about 20 g/L, or from about 1 g/L to about 5 g/L, or from about 2 g/L to about 5 g/L, or about 1 g/L to about 10 g/L, or from about 2 g/L to about 10 g/L, or from about 5 g/L to about 20 g/L, or from about 5 g/L to about 10 g/L, or from about 4 g/L to about 10 g/L). In some embodiments, the composition comprises verapamil in an amount of about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 16 g/L, about 17 g/L, about 18 g/L, about 19 g/L or about 20 g/L In some embodiments, the composition comprises verapamil in an amount of about 4 g/L, about 5 g/L, about 6 g/L, or about 7 g/L. In some embodiments, the composition does not include a calcium channel blocker. In some embodiments, the composition does not include verapamil.

In some embodiments, the composition further comprises a sodium channel blocker in an amount ranging from 0.4 g/L to about 45 g/L (or from about 1 g/L to about 10 g/L, or from about 0.4 g/L to about 10 g/L, or from about 1 g/L to about 5 g/L, or from about 5 g/L to about 20 g/L, or from about 10 g/L to about 30 g/L, or from about 10 g/L to about 45 g/L). In some embodiments, the composition comprises a sodium channel blocker in an amount of about 0.4 g/L, about 0.8 g/L, about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 41 g/L, about 42 g/L, about 43 g/L, about 44 g/L, or about 45 g/L. Exemplary sodium channel blockers include, but are not limited to, carbamazepine, benzocaine, bupivacaine, lidocaine, procaine, mepivacaine, and mexiletine. In some embodiments, the sodium channel blocker is mexiletine.

In some embodiments, the composition comprises mexiletine in an amount ranging from 0.4 g/L to about 45 g/L (or from about 1 g/L to about 10 g/L, or from about 0.4 g/L to about 10 g/L, or from about 1 g/L to about 5 g/L, or from about 5 g/L to about 20 g/L, or from about 10 g/L to about 30 g/L, or from about 10 g/L to about 45 g/L). In some embodiments, the composition comprises a mexiletine in an amount of about 0.4 g/L, about 0.8 g/L, about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 41 g/L, about 42 g/L, about 43 g/L, about 44 g/L, or about 45 g/L. In some embodiments, the composition does not include mexiletine.

In some embodiments, the composition further comprises a red blood cell (RBC) stabilizer. In some embodiments, the RBC stabilizer is a cyclodextrin, Doxycycline, Polyethylene Glycol, Sulfasalazine, Polyvinylpyrrolidone, Curcumin, Magnesium Gluconate, Homocysteine, Methyl Cellulose (MC), 6-Aminocaproic acid, Ethyl Cellulose, Aprotinin, Hydroxyethyl Cellulose, Doxycycline, Hydroxypropyl Cellulose, Minocycline HCl, Dextrin, Nicotinamide, Dextran, Chitosan, Polyethylene Oxide, Lysine, Poly Ethyl Oxazoline, Glyceraldehyde, Ficolls, Phytic Acid, α-Cyclodextrin, b-Sitoserol, ß-Cyclodextrin, C-AMP, Y⁻-Cyclodextrin, Poly Lysine, Gelatins, Biochanin A, Sugars (e.g., sucrose, mannitol, lactose, trehalose), Sulfasalazine, Hydroxypropyl Methyl Cellulose, Demeclocycline, Hydroxyethyl Methyl Cellulose, Chlortetracycline, Oxytetracycline, Cyclohexamide, Rifampicin, Soy Milk, soybean based protease inhibitor, Suramin, N-Butyric Acid, Penicillamine, N-Acetyl Cysteine, Benzamidine, AEBSF, Alpha-2 Macroglobulin, or combinations thereof. It is contemplated that one or more of the foregoing compounds can be substituted for cyclodextrin in a composition of the disclosure. In some embodiments, the RBC stabilizer is a cyclodextrin. Exemplary cyclodextrins include, but are not limited to, α-cyclodextrin, β-cyclodextrin and γ-cyclodextrin.

In some embodiments, the composition comprises α-cyclodextrin in an amount ranging from about 10 g/L to about 200 g/L, about 10 g/L to about 150 g/L, about 10 g/L to about 100 g/L, about 10 g/L to about 75 g/L, about 10 g/L to about 50 g/L, about 15 g/L to about 30 g/L. In some embodiments, the composition comprises α-cyclodextrin in an amount of about 10 g/L, about 15 g/L, about 20 g/L, about 25 g/L about 30 g/L, about 35 g/L, about 40 g/L, about 45 g/L, about 50 g/L, about 55 g/L, about 60 g/L, about 70 g/L, about 75 g/L, about 80 g/L, about 85 g/L, about 90 g/L, about 95 g/L, about 100 g/L, about 125 g/L, about 150 g/L, about 175 g/L, or about 200 g/L. In some embodiments, the composition comprises α-cyclodextrin in an amount of about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L or about 30 g/L.

In some embodiments, the composition may include one or several preservatives that are selected from paraben, boric acid, polylysine, procline, LiCl, tertiary amide, dextran sulfate, nitrate sulfate, glycerol, and polypol.

In exemplary embodiments, the composition is substantially non-toxic and/or chemically inert with respect to the blood sample and any components thereof, e.g., cells, proteins, nucleic acids, exosomes, and the like. In various aspects, the composition is substantially free of formaldehyde, paraformaldehyde, guanidinium salts, sodium dodecyl sulfate (SDS), or any combination thereof. In some aspects, the composition is substantially free of formaldehyde. For instance, the composition comprises formaldehyde in an amount that is less than or about 50,000 ppm. The composition in some aspects comprises less than about 20 parts per million (ppm) of formaldehyde. The composition may contain less than about 15 parts per million (ppm) of formaldehyde. The composition may contain less than about 10 parts per million (ppm) of formaldehyde. The composition may contain less than about 5 parts per million (ppm) of formaldehyde. The composition may contain at least about 0.1 parts per million (ppm) to about 20 ppm of formaldehyde. The composition may contain at least about 0.5 parts per million (ppm) to about 15 ppm of formaldehyde. The composition may contain at least about 1 parts per million (ppm) to about 10 ppm of formaldehyde.

As further described herein, the composition is substantially free of proteolytic enzyme inhibitors which do not also function as an AC. As used herein, the term “proteolytic enzyme inhibitors” refer to any agent, chemical (e.g., small molecule) or biological, naturally occurring or synthetic, which inhibits a protease or proteinase. Proteases are classified by their mechanism of action, and include for example, serine proteases, cysteine (thiol) proteases, aspartic proteases, metalloproteases, endoproteases, trypsin-like proteases, chymotrypsin-like proteases, caspase-like proteases, elastase-like proteases. In various aspects, the proteolytic enzyme inhibitor reduces the activity of one or more of these proteases. Proteolytic enzyme inhibitors are known in the art and include, but are not limited to: alpha-2-macroglobulin, 4-(2-Aminoethyl)benzenesulfonyl fluoride (AEBSF), Amidinophenylmethanesulfonyl fluoride hydrochloride; (APMSF), amastatin, antipain, aprotinin, bestatin, chymostatin, diprotin A, diprotin B, EDTA, E-64, egg white cystatin, egg white ovostatin, elastatinal, galardin, indoleacetic acid (IAA), leupeptin, trypsin inhibitors (e.g., soybean trypsin inhibitor), nelfinavir mesylate, pepstatin (e.g., pepstatin A), phenylmethylsulfonyl fluoride (PMSF), phosphoramodon, 1,10-phenanthroline, pancreatic protease inhibitor, 4-Tosyl-L-lysyl-chloromethane hydrochloride (TLCK), Tosyl phenylalanyl chloromethyl ketone (TPCK), VdLP FFVdL, and any combination thereof. The composition is substantially free of these inhibitors or any combination thereof, including any combination of proteolytic enzyme inhibitors sold by commercial suppliers and referred to as a “protease inhibition cocktails”. Mixtures, combinations, or cocktails of protease inhibitors are also known in the art, including Roche complete tablets, Roche complete ULTRA (EDTA-free) protease inhibitor cocktail tablet, Calbiochem protease inhibitor cocktail, Halt Protease Inhibitor Cocktail, G-Biosciences FOCUS™ ProteaseArrest™, Recom ProteaseArrest™.

Proteolytic enzyme inhibitors which also function as an AC include, but are not limited to, EDTA and EGTA.

In exemplary embodiments, the composition comprises citrate-theophylline-adenosine-dipyrridamole (CTAD), imidazolidinyl urea and α-cyclodextrin. In some embodiments, the protective agent comprises imidazolidinyl urea at a concentration of about 100 g/l to about 400 g/l. In some embodiments, the composition comprises citric acid at a concentration of about 10 g/l to about 50 g/l citric acid. In some embodiments, the composition comprises theophylline at a concentration of about 1 g/l to about 20 g/l. In some embodiments, the composition comprises adenosine at a concentration of about 1 g/l to about 20 g/l. In some embodiments, the composition comprises dipyridamole at a concentration of about 0.05 g/l to about 20 g/l. In some embodiments, the composition comprises α-cyclodextrin at a concentration of about 10 g/l to about 50 g/l α-cyclodextrin. In some embodiments, the composition consists essentially of (i) about 100 g/l to about 400 g/l imidazolidinyl urea (ii) about 10 g/l to about 50 g/l citric acid; (iii) about 1 g/l to about 20 g/l theophylline; (iv) about 1 g/l to about 20 g/l adenosine; (v) about 0.05 g/l to about 20 g/l dipyridamole; and (vi) about 10 g/l to about 50 g/l α-cyclodextrin.

In exemplary embodiments, the composition comprises citrate-theophylline-adenosine-dipyrridamole (CTAD), and imidazolidinyl urea. In some embodiments, the composition comprises imidazolidinyl urea at a concentration of about 100 g/l to about 400 g/l. In some embodiments, the composition comprises citric acid at a concentration of about 10 g/l to about 50 g/l citric acid. In some embodiments, the composition comprises theophylline at a concentration of about 1 g/l to about 20 g/l. In some embodiments, the composition comprises adenosine at a concentration of about 1 g/l to about 20 g/l. In some embodiments, the composition comprises dipyridamole at a concentration of about 0.05 g/l to about 20 g/l. In some embodiments, the composition consists essentially of (i) about 100 g/l to about 400 g/l imidazolidinyl urea (ii) about 10 g/l to about 50 g/l citric acid; (iii) about 1 g/l to about 20 g/l theophylline; (iv) about 1 g/l to about 20 g/l adenosine; and (v) about 0.05 g/l to about 20 g/l dipyridamole.

In exemplary embodiments, the composition comprises is citrate-phosphate-dextrose-adenine (CPDA), imidazolidinyl urea and α-cyclodextrin. In some embodiments, the composition comprises imidazolidinyl urea at a concentration of about 100 g/l to about 400 g/l. In some embodiments, the composition comprises citric acid at a concentration of about 10 g/l to about 50 g/l citric acid. In some embodiments, the composition comprises trisodium citrate at a concentration of about 10 g/L to about 200 g/L. In some embodiments, the composition comprises monobasic sodium phosphate at a concentration of about 10 g/l to about 200 g/l. In some embodiments, the composition comprises dextrose at a concentration of about 50 g/l to about 300 g/l. In some embodiments, the composition comprises adenine at a concentration of about 0.05 g/l to about 20 g/l. In some embodiments, the composition comprises α-cyclodextrin at a concentration of about 10 g/l to about 50 g/l α-cyclodextrin. In some embodiments, the composition consists essentially of (i) about 100 g/l to about 400 g/l imidazolidinyl urea, (ii) about 10 g/l to about 50 g/l citric acid; (iii) about 10 g/l to about 200 g/l trisodium citrate; (iv) about 50 g/l to about 300 g/l dextrose; (iv) (v) 10 g/l to about 200 g/l about monobasic sodium phosphate; (vi) about 0.05 g/l to about 20 g/l adenine; and (vii) about 10 g/l to about 50 g/l α-cyclodextrin.

In exemplary embodiments, the composition comprises citrate-phosphate-dextrose-adenine (CPDA), and imidazolidinyl urea. In some embodiments, the composition comprises imidazolidinyl urea at a concentration of about 100 g/l to about 400 g/l. In some embodiments, the composition comprises citric acid at a concentration of about 10 g/l to about 50 g/l citric acid. In some embodiments, the composition comprises trisodium citrate at a concentration of about 10 g/L to about 200 g/L. In some embodiments, the composition comprises monobasic sodium phosphate at a concentration of about 10 g/l to about 200 g/l. In some embodiments, the composition comprises dextrose at a concentration of about 50 g/l to about 300 g/l. In some embodiments, the composition comprises adenine at a concentration of about 0.05 g/l to about 20 g/l. In some embodiments, the composition consists essentially of (i) about 100 g/l to about 400 g/l imidazolidinyl urea, (ii) about 10 g/l to about 50 g/l citric acid; (iii) about 10 g/l to about 200 g/l trisodium citrate; (iv) about 50 g/l to about 300 g/l dextrose; (v) 10 g/l to about 200 g/l about monobasic sodium phosphate; (vi) about 0.05 g/l to about 20 g/l adenine.

Compositions comprising an AC, an AR, and a red blood cell (RBC) stabilizer described herein are also contemplated. In some embodiments, the composition comprises citrate-theophylline-adenosine-dipyrridamole (CTAD), imidazolidinyl urea and α-cyclodextrin. In some embodiments, the composition comprises citrate-theophylline-adenosine-dipyrridamole (CTAD) and imidazolidinyl urea. In some embodiments, the composition comprises citrate-phosphate-dextrose-adenine (CPDA), imidazolidinyl urea and α-cyclodextrin. In some embodiments, the composition comprises citrate-phosphate-dextrose-adenine (CPDA) and imidazolidinyl urea.

In some embodiments, the composition comprises citric acid, trisodium citrate, dextrose, imidazolidinyl urea, alpha-cyclodextrin, verapamil hydrochloride, and mexiletine hydrochloride. In some embodiments, the composition is substantially free of any proteolytic enzyme inhibitors which do not also function as an AC. In some embodiments, the composition comprises citric acid at a concentration of about 10 g/L to about 50 g/L citric acid, trisodium citrate at a concentration of about 10 g/L to about 200 g/L trisodium citrate, dextrose at a concentration of about 50 g/L to about 300 g/L dextrose, imidazolidinyl urea at a concentration of about 100 g/L to about 1000 g/L imidazolidinyl urea, alpha-cyclodextrin at a concentration of about 10 g/L to about 50 g/L α-cyclodextrin, verapamil at a concentration of about 2 g/L to about 10 g/L verapamil, and mexiletine at a concentration of about 0.4 g/L to about 45 g/L mexiletine.

In some embodiments, the composition comprises 10 g/L to 20 g/L citric acid, 50 g/L to 70 g/L trisodium citrate, 65 g/L to 85 g/L dextrose, 140 g/L to 160 g/L imidazolidinyl urea, 1 g/L to 10 g/L verapamil and 1 g/L to 10 g/L mexiletine (“Streck MO”). In some embodiments, the composition further comprises 10 g/L to 20 g/L α-cyclodextrin.

In some embodiments, the composition comprises 10 g/L to 30 g/L citric acid, 50 g/L to 150 g/L trisodium citrate, 65 g/L to 160 g/L dextrose, 140 g/L to 160 g/L imidazolidinyl urea, 15 g/L to 30 g/L α-cyclodextrin, 1 g/L to 10 g/L verapamil.

In some embodiments, the composition comprises 10 g/L to 40 g/L citric acid, 50 g/L to 150 g/L trisodium citrate, 65 g/L to 160 g/L dextrose, 140 g/L to 160 g/L imidazolidinyl urea, 15 g/L to 30 g/L α-cyclodextrin, and 1 g/L to 10 g/L mexiletine. In some embodiments, the composition comprises 10 g/L to 40 g/L citric acid, 50 g/L to 150 g/L trisodium citrate, 65 g/L to 160 g/L dextrose, 140 g/L to 160 g/L imidazolidinyl urea, 10 g/L to 30 g/L α-cyclodextrin, and 1 g/L to 10 g/L mexiletine, wherein the composition does not include verapamil.

In some embodiments, the composition comprises 10 g/L to 30 g/L citric acid, 50 g/L to 150 g/L trisodium citrate, 65 g/L to 160 g/L dextrose, 140 g/L to 160 g/L imidazolidinyl urea, 15 g/L to 30 g/L α-cyclodextrin, and 1 g/L to 10 g/L verapamil. In some embodiments, the composition comprises 10 g/L to 20 g/L citric acid, 50 g/L to 150 g/L trisodium citrate, 65 g/L to 160 g/L dextrose, 140 g/L to 160 g/L imidazolidinyl urea, 10 g/L to 30 g/L α-cyclodextrin, and 1 g/L to 10 g/L verapamil, wherein the composition does not include mexiletine.

In some embodiments, the composition comprises 18 g/L to 23 g/L citric acid, 92 g/L to 105 g/L trisodium citrate, 105 g/L to 115 g/L dextrose, 140 g/L to 160 g/L imidazolidinyl urea, 18 g/L to 23 g/L α-cyclodextrin, and 5 g/L to 8 g/L verapamil, wherein the composition does not include mexiletine. (“Streck MH1.5”)

In some embodiments, the composition comprises 12 g/L to 17 g/L citric acid, 62 g/L to 70 g/L trisodium citrate, 68 g/L to 78 g/L dextrose, 140 g/L to 160 g/L imidazolidinyl urea, 10 g/L to 17 g/L α-cyclodextrin, and 3 g/L to 5 g/L verapamil, wherein the composition does not include mexiletine. (“Streck MH”)

In some embodiments, the composition comprises 25 g/L to 35 g/L citric acid, 125 g/L to 135 g/L trisodium citrate, 140 g/L to 150 g/L dextrose, 140 g/L to 160 g/L imidazolidinyl urea, 25 g/L to 35 g/L α-cyclodextrin, and 5 g/L to 10 g/L mexiletine, wherein the composition does not include verapamil. (“Streck M2”)

In exemplary aspects, the composition further comprises one or more preservative agents, enzyme inhibitors, metabolic inhibitors, or a combination thereof. The one or more enzyme inhibitors in some aspects is diethyl pyrocarbonate, ethanol, aurintricarboxylic acid (ATA), glyceraldehydes, sodium fluoride, formamide, vanadyl-ribonucleoside complexes, macaloid, heparin, hydroxylamine-oxygen-cupric ion, bentonite, ammonium sulfate, dithiothreitol (DTT), beta-mercaptoethanol, cysteine, dithioerythritol, tris (2-carboxyethyl) phosphene hydrochloride, a divalent cation (such as Mg⁺², Mn⁺², Zn⁺², Fe⁺², Ca⁺², Cu⁺²), or any combination thereof. In exemplary instances, the composition comprises one or more nuclease inhibitors, e.g., DNAse inhibitor or RNase inhibitor. The one or more metabolic inhibitors in certain aspects is glyceraldehyde, dihydroxyacetone phosphate, glyceraldehyde 3-phosphate, 1,3-bisphosphoglycerate, 3-phosphoglycerate, 2-phosphoglycerate, phosphoenolpyruvate, pyruvate or glycerate dihydroxyacetate, sodium fluoride, K₂C₂O₄ or a combination thereof. In exemplary aspects, the composition does not comprise a preservative agent, enzyme inhibitor, metabolic inhibitor, described above.

### Blood Collection Devices

In some embodiments, the composition is present in a blood collection device. In some embodiments, the blood collection device is a collection tube (BCT). In some embodiments, the blood collection device is a micro collection blood collection tube or a capillary blood collection tube (e.g., BD Microtainer® Blood Collection Tubes).

The composition in exemplary aspects is added to a blood sample. In other aspects, the composition is present in a blood collection device and the blood sample is added to the blood collection device comprising the composition. Suitable blood collection devices are known in the art and include those described in International Patent Publication No. WO2018145005 and U.S. Pat. No. 9,657,227.

The blood collection devices may be made of any suitable, non-toxic, chemically-inert material, such as a plastic, glass, silica, carbon, or a combination thereof. In exemplary aspects, the blood collection device is made of a material which minimizes adhesion or adherence of cells or proteins or other components of the blood sample. In some aspects, the blood collection device is made of a transparent material. In various instances, the blood collection device is composed of a material comprising polypropylene, polystyrene, or glass (e.g., borosilicate glass, flint glass, aluminosilicate glass, soda lime glass, lead or quartz glass). In exemplary instances, the tube is composed of a material comprising a cyclic polyolefin, e.g., a cyclic polyolefin copolymer or cyclic polyolefin polymer. In exemplary aspects, the materials may be stable at a temperature of about −100° C. to about 50° C. (e.g., 2° C. to about 30° C.) and thus may be suitable for storing samples in a freezer, refrigerator, heater, heated incubator, heated water bath, or at room temperature.

The composition may be present in a blood collection device in an amount less than about 10% by volume of the blood collection device but greater than about 0.1% by volume. The composition may be present in the blood collection device in an amount less than about 5% by volume of the blood collection device but greater than about 0.1% by volume of the blood collection device. The composition may be present in the blood collection device in an amount less than about 3% by volume of the blood collection device but greater than about 0.1% by volume of the blood collection device.

In some embodiments, the blood collection device volume:composition volume ratio ranges from 5:1 to 40:1, or 10:1 to 40:1 (e.g., 5:1, 6:1, 7:1, 8:1, 9:1, 10:1, 11:1, 12:1, 13:1, 14:1, 15:1, 20:1, 25:1, 30:1, 35:1, or 40:1).

The blood collection device may have any geometry or suitable shape for containing and storing a liquid. In exemplary aspects, the tube is substantially cylindrical in shape with one closed end and one open end. In exemplary aspects, the tube comprises an enclosed base, a coextensive elongated side wall extending from the base and terminating at an open end, such that a hollow chamber having an inner wall is defined. In certain aspects, the hollow chamber is configured for collecting a blood sample. In various aspects, at least the elongated side wall of the blood collection device is made of a material including a thermoplastic polymeric material having a high moisture barrier and low moisture absorption rate, and optical transparency to enable viewing a sample within the tube and chemical resistance. The closed end or enclosed base in some aspects is round-bottomed or U-shaped, conical or V-shaped. The open end in various instances comprises a series of threads suitable for fitting a screw cap for temporary closure. In alternative aspects, the open end does not comprise a series of threads. In various instances, the open end may be fitted with a stopper or a cap. In exemplary aspects, the closed end or the enclosed base of the blood collection device is flat. In various instances, at least a portion of the elongated side wall of the blood collection device tapers to a point located at or within the enclosed base or closed end. The blood collection device in some aspects has an outer diameter, as measured at the coextensive elongated side wall adjacent the open end, to length (D×L) dimension of about 13 mm×75 mm. The blood collection device in some aspects has an outer diameter, as measured at the coextensive elongated side wall adjacent the open end, to length (D×L) dimension of about 16 mm×100 mm. In alternative instances, the elongated side wall of the blood collection device does not taper to a point.

In various aspects, the volumetric capacity of the blood collection device is at least or about 0.01 mL, at least or about 0.10 mL, at least or about 0.2 mL, at least or about 0.25 mL, at least or about 0.5 mL, at least or about 1 mL, at least or about 1.5 mL, at least or about 2 mL, at least or about 2.5 mL, at least or about 3 mL, at least or about 3.5 mL, at least or about 4 mL, at least or about 4.5 mL, or at least or about 5 mL, and optionally up to about 350 mL, up to about 300 mL, up to about 250 mL, up to about 200 mL, up to about 150 mL, or up to about 100 mL. In some instances, the tube can hold about 1 mL to about 45 mL, about 1 mL to about 40 mL, about 1 mL to about 35 mL, about 1 mL to about 30 mL, about 1 mL to about 25 mL, about 1 mL to about 20 mL, about 1 mL to about 15 mL, about 1 mL to about 10 mL. In various aspects, the volumetric capacity is about 0.25 mL to about 1 mL, about 5 mL to about 40 mL, about 5 mL to about 30 mL, about 5 mL to about 20 mL, about 5 mL to about 15 mL, optionally, about 5 mL, about 6 mL, about 7 mL, about 8 mL, about 9 mL, about 10 mL, about 11 mL, about 12 mL, about 13 mL, about 15 mL, about 16 mL, about 17 mL, about 18 mL, about 19 mL, about 20 mL.

In one general sense, the present teachings pertain to a sample collection container sized and configured to secure an appropriate amount of a biological sample for multi-omic analysis (e.g., analysis of such as genomics, proteomics, transcriptomics, lipidomics, and/or metabolomics), and including within the container a coating including a stabilizing reagent on an interior surface of the container. The biological sample may be blood, urine, saliva, mucus, cerebrospinal fluid, fecal matter, amniotic fluid, or other fluidic discharge from a human or animal, and/or any constituent of the above. The coating (which may be a dried coating or spraycoating) may have a predefined pattern that spans two or three dimensions. For example, the predefined pattern may include a pattern possessing at least one continuous film. The continuous film may be continuous over a predefined length. The predefined pattern may include a plurality of coating particles. The predefined pattern may include a continuous film portion, a plurality of spaced continuous films, a plurality of discrete coating particles, a liquid or solid pellet, or any combination thereof.

The coating may include a stabilizing reagent in a form that is capable of dissolving in the presence of, and upon contact with at least a portion of the liquid biological sample. The dried coating may include a stabilizing reagent in a form that is capable of dispersing (e.g., after dissolution) within the biological sample for causing stabilization of any target (e.g., proteins, nucleic acids, extracellular vesicles, white blood cells, circulating tumor cells, circulating rare cells, platelets, or any combination thereof) intended for multi-omic analysis.

### Methods

The present disclosure also methods for stabilizing a blood sample comprising contacting a blood sample with a composition described herein, wherein the composition stabilizes fractions within the blood sample. In some embodiments, the blood sample is contacted with the composition by adding the blood sample to a blood collection device comprising the composition. In some embodiments, the blood sample is directly drawn from a subject into a blood collection device comprising the composition.

In alternative aspects, the blood sample is contacted with the composition by adding the composition to the blood sample. In some embodiments, the methods described herein further comprise isolating a cellular fraction of the blood sample.

In some embodiments, the methods disclosed herein further comprise a step of isolating a fraction comprising proteins form the blood sample. In some embodiments, the fraction comprises plasma of the blood sample. In some embodiments, the fraction is a plasma fraction (e.g., a fraction that is substantially free of cells, e.g., substantially free of red blood cells, white blood cells, platelets). In some embodiments, the methods further comprise isolating one or more of proteins, DNA, RNA and extracellular vesicles from the plasma fraction. In some embodiments, the methods further comprise isolating cell free nucleic acids (cfRNA, cfRNA or both) from the plasma fraction. In some embodiments, the methods further comprise isolating plasma proteins, cytokines and peptides from the plasma fraction.

In some embodiments, the fraction comprises cells of the blood sample (i.e., cellular fraction of the blood sample). In some embodiments, the cellular fraction comprises (or consists essentially of) rare blood cells, including but not limited to, rare white blood cells (e.g., eosinophils, Jacobsen et al., Blood, 120:3882-3890, 2012); circulating tumor cells (CTCs), fetal circulating cells, rare white blood cells, and other rare circulating nuclear cells. In some embodiments, the cellular fraction comprises (or consists essentially of) circulating nuclear cells, which includes but not limited to white blood cells (e.g., peripheral blood mononuclear cells, nucleated red blood cells). In some embodiments, the cellular fraction is free of red blood cells, white blood cells, platelets, or a combination thereof. The cellular fraction in some instances is free of plasma proteins. In some embodiments, the methods described herein comprise lysing cells of the cellular fraction to obtain a protein sample suitable for proteomic analysis.

The isolating step of methods described herein, in some aspects, comprise a centrifugation step. For example, the centrifugation step may be such that the centrifugation step yields a cell pellet and a cell-free supernatant. In some embodiments, the isolating step comprises isolating plasma by, e.g., centrifuging the blood sample at about 2000 g for about 15 minutes. Optionally, the isolated plasma is further centrifuged to obtain clarified plasma. In some embodiments, the methods described herein may comprise centrifuging the blood sample at about 2000 g for about 15 minutes (optionally at room temperature) to obtain a supernatant comprising isolated plasma, followed by centrifuging the supernatant comprising the isolated plasma at about 16,000 g for about 10 minutes (optionally at room temperature) to obtain a supernatant comprising clarified plasma. In some embodiments, the plasma (e.g., the clarified plasma) is further processed. For example, the clarified plasma may be depleted of proteins that are present in plasma at a relatively high concentration. In exemplary instances, the clarified plasma is depleted of immunoglobulins, albumin, or a combination of both. Methods of depletion are known in the art and include use of spin trap columns.

In some embodiments, the isolating step of the methods disclosed herein comprises one or more chromatography steps, electrophoretic separation steps, immunoprecipitation steps, or a combination thereof. Suitable techniques for isolating fractions comprising proteins from blood samples are known in the art.

In some embodiments, the isolating step of the methods disclosed herein comprises a cell sorting step, e.g., a fluorescence activated cell sorting (FACS) step. The cell sorting step, in some embodiments, is based on expression of a cell surface protein on some cells, or a lack of expression of a cell surface protein on some cells.

In some embodiments, the isolating step yields a protein sample comprising substantially the same types and amounts of proteins (e.g., intact proteins) as in the blood sample upon collection into the blood collection device. In other words, the protein sample is substantially the same as the original blood sample in terms of the proteins present in the sample and the amount of each protein. Also, in some embodiments, the protein sample has little to substantially no loss of proteins through protein degradation or protein aggregation. In various aspects, the protein sample has little to substantially no contaminant protein products. In exemplary embodiments, the isolating step yields a protein sample comprising less than about 25% contaminant protein products as measured by high performance liquid chromatography mass spectrometry (HPLC-MS). As used herein, the term “contaminant protein products” refers to unwanted protein products including but not limited to protein fragments, intact intracellular proteins, aggregates of whole proteins and/or protein fragments, and the like which result from degradation, aggregation (optionally via protein-protein intramolecular association forces), protein self-association reactions, and the like. Chromatographic techniques, such as HPLC, can detect the amount of contaminant proteins in a given sample. In exemplary aspects, the protein sample comprises less than about 20% contaminant protein products, less than about 15% contaminant protein products, less than about 10% contaminant protein products, or less than about 5% contaminant protein products, as measured by HPLC-MS. In some aspects, the protein sample comprises less than about 4% contaminant protein products, less than about 3% contaminant protein products, or less than about 2% contaminant protein products, as measured by HPLC-MS.

The methods described herein may comprise repeating any one of the above-described step(s) and/or may comprise additional steps, aside from those described above. For example, the presently disclosed methods may further comprise steps to further process the sample prior to isolating the fraction comprising protein to yield the protein sample. In various instances, the method comprises one or more centrifuging steps to isolate plasma and/or obtain clarified plasma, as described above. In various aspects, the method comprises one or more protein separation steps, e.g., chromatographic, electrophoretic or immunoprecipitation steps. In exemplary aspects, the method comprises depleting the sample of unwanted high concentration proteins, e.g., albumin and/or immunoglobulins. In some aspects, the method comprises one or more of: (a) adding a digestion enzyme, a reducing agent, an alkylating agent, to the sample; (b) identifying proteins present in the sample; (c) quantitating total and individual protein concentration of the sample or an aliquot thereof; and/or (d) labeling proteins or a subset thereof with a tag. Optionally, the digestion enzyme is trypsin. In some aspects, the reducing agent comprises urea or dithiothreitol (DTT) or both. In certain instances, the alkylating agent comprises iodoacetamide (IAA), or a combination thereof.

In various aspects, the methods described herein further comprises transporting the mixture in a sealed container to a laboratory for analysis. Optionally, the sealed container is a sealed collection device comprising the composition. In some aspects, the transport to the laboratory requires storing the mixture in the sealed container for at least 24 hours, at least 36 hours, at least 48 hours, at least 60 hours, at least 72 hours, about 84 hours, at least 96 hours or more. In some aspects, the transporting step entails storing the mixture in the sealed container for a storage period for at least about 5 days, at least about 6 days, at least 7 days, or more. In various aspects, the transporting step entails storing the mixture in the sealed container for a storage period at refrigerated temperatures, e.g., about 2° C. to about 8° C., or at temperatures above these temperatures, e.g., about 2° C. to about 30° C. about 10° C. to about 15° C., or at an ambient temperature e.g., about 15° C. to about 30° C., about 20° C. to about 25° C., about 20° C. to about 30° C. In various aspects, after the transporting step, the mixture is suitable for proteomic analysis as evidenced by the slope of the best fit line of a line graph of the number of proteins in the protein sample yielded from step (b) plotted as a function of storage time being closer to 0 compared to the slope of the best fit line of a line graph of the number of proteins in a control blood sample not contacted with a composition and/or the number of plasma proteins and/or peptides present in the protein sample following storage for at least 48 hours being within about 10% (e.g., about 10%, about 9%, about 8%, about 7%, about 6%, about 5%, about 4%, about 3%, about 2%, about 1% or less) of the number of plasma proteins and/or peptides present in the protein sample within about 0 hours to about 4 hours of collecting the blood sample from a subject. In some embodiments, number of plasma proteins and/or peptides present in the protein sample following storage for at least 48 hours is >90% of the number of plasma proteins and/or peptides present in the protein sample within about 0 hours to about 4 hours of collecting the blood sample from a subject.

In exemplary aspects, the method comprises conducting proteomic (or peptidomic) analysis. In this regard, the methods may comprise a step of analyzing the proteins using one or more mass spectrometry-based proteomic methods. Suitable methods of proteomic analysis are known in the art, including but not limited to turbidometry, electrophoresis (e.g., capillary electrophoresis, one-dimensional or two-dimensional gel electrophoresis, polyacrylamide gel electrophoresis (PAGE), differential gel electrophoresis (DIGE)), immunoaffinity-based techniques (e.g., Enzyme linked immunosorbent assay (ELISA), sandwich ELISA, competitive ELISA, immunoprecipitation, immunoelectrophoresis, radioimmunoassay), mass spectrometry (e.g., electrospray ionization (ESI)-MS/MS, matrix assisted laser dissociation spectrometry (MALDI)-TOF MS, laser microdissection (LMD)/MS, liquid chromatograph coupled mass spectrometry (LC-MS/MS)), high performance liquid chromatography (HPLC) among other quantitative proteomic techniques (e.g., iTRAQ, ICAT, SILAC), multidimensional protein identification technology (MudPIT), reverse phase protein array, SOMAmers Technology, SELDI, SCX, and the like. See, e.g., Ahmed et al., 2014, supra. In certain aspects, the mass spectrometry-based proteomic methods is a targeted mass spectrometry. In some aspects, the mass spectrometry experiment utilizes parallel reaction monitoring (PRM), selected reaction monitoring (SRM), selected ion monitoring (SIM), or multiple reaction monitoring (MRM). In alternative aspects, the mass spectrometry is a not targeted mass spectrometry. Optionally, the mass spectrometry experiment utilizes data-dependent acquisition (DDA), data independent acquisition (DIA), or labeled quantitation (e.g., tandem mass tag (TMT)) mass spectrometry. Peptidomic analysis employs many proteomics techniques but with a different target. Rather than examining a sample for which intact proteins are present (proteomics), peptidomics examines which endogenous protein fragments are present.

In exemplary aspects, the method further comprises carrying out the proteomic analysis and a genomic analysis. Suitable techniques of analyzing the genomic content of a sample are known in the art. See, e.g., Chromosomal Microarray (CMA), linkage analysis, whole exome sequencing (WES), next generation DNA sequences (NGS), and the like.

In exemplary aspects, the method further comprises carrying out analysis on the nucleic acid components of a blood sample. Suitable techniques for analyzing nucleic acids in a sample, include but are not limited to, Polymerase chain reaction (PCR), Reverse Transcription-PCR, real-time RT-PCR, digital PCR, digital droplet PCR, next generation sequencing, TapeStation, Electrophoresis, Bioanalyzer, hybridization-based target capture or detection, DNA methylation analysis, immunoassay, mass spectrometry analysis, single cell sequencing, etc.

In exemplary aspects, the method further comprises carrying out analysis on the extracellular vesicle components of a blood sample. Suitable techniques include but not limited to study EV profile and EV cargo with flow cytometry, Chip-based EV capture and analysis, conducting genomic, proteomic, transcriptomic, lipidomic and metabolic analysis on EV cargoes, etc.

In exemplary aspects, the method further comprises carrying out analysis on the cellular components of a blood sample. Exemplary cellular components include but not limited to peripheral blood mononuclear cell, circulating fetal cells, circulating tumor cells, etc.

### Storage Stability

Without being bound to a particular theory, the composition allows for stabilization of the blood sample, reducing or preventing cell lysis and subsequent release of cellular proteins into the sample. Advantageously, the protein sample isolated from the blood sample is characterized by minimized levels of contaminant protein products, as further described herein. Due to the enhanced stability of the blood sample imparted by the composition, the blood sample is capable of longer periods of storage at both refrigerated temperatures and at higher temperatures, e.g., temperatures above 4° C. Surprisingly, the blood sample in contact with the composition may be stored for greater than 48 hours and up to 7 days or even longer. The stability allows for the blood sample in contact with the composition to be stored for at least 48 hours at 20° C., for example, and the stability of the blood sample may be evidenced by the low amounts of contaminant protein products after the storage period.

Accordingly, in some aspects, prior to the step of isolating a fraction or cellular fraction, the mixture had been stored for at least 48 hours, for at least 48 hours but less than 7 days or for at least 48 hours but less than 14 days. Optionally, prior to the step of isolating a fraction or cellular fraction, the mixture had been stored for at least 48 hours at a temperature greater than 4° C., optionally, at a temperature of about 20° C. to about 25° C.

Also, accordingly, in various instances, the method comprises storing the mixture prior to the step of isolating a fraction or cellular fraction. In some aspects, the method comprises storing the mixture in the BCT for at least 48 hours, for at least 48 hours but less than 7 days, or for at least 48 hours but less than 14 days prior to the step of isolating a fraction or cellular fraction.

The storage stability of the mixture imparted by the composition advantageously allows for subsequent analysis to be carried out on the blood sample at a much later time after the blood sample has been collected, e.g., drawn from the subject. Such storage stability avoids the problems associated with freezing and thawing the protein sample prior to the proteomic analysis.

### Reduced Cell Lysis and Contaminating Cellular Proteins Upon Storage

The composition allows for stabilization of the blood sample, reducing or preventing cell lysis and subsequent release of cellular proteins into the sample. The protein sample yielded by the presently disclosed methods is advantageously characterized by reduced or decreased cell lysis. While a minimal or base level of cell lysis occurs due to the shear of collecting the blood from the subject, for example, the amount of cell lysis increases over time, e.g., upon storage at refrigerated temperatures or higher temperatures. As a result of the composition imparting stability, the protein sample yielded in the method is suitable for proteomic analysis due to the reduced level in cell lysis. In some aspects, the reduced level in cell lysis is a reduction of at least about 10% (e.g., at least about 20%, at least about 30%, at least about 40%, at least about 50%, at least about 60%, at least about 70%, at least about 80% at least about 90%, or more) relative to the level of cell lysis of a control protein sample obtained from an isolated fraction of a blood sample collected in a blood collection tube without the composition described herein. In some aspects, the reduced level in cell lysis is a reduction of at least about 10% (e.g., at least about 20%, at least about 30%, at least about 40%, at least about 50%, at least about 60%, at least about 70%, at least about 80% at least about 90%, or more) relative to the level of cell lysis of a control protein sample obtained from an isolated fraction of a blood sample collected in a blood collection tube without a composition (e.g., comprising only EDTA), following storage of the mixture for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring cell lysis are known in the art and include for example, measurement of cell lysis by light scattering (Valenzeno and Trank, Photochemistry and Photobiology 42(3): 335-339 (1985)) and measurement using cell dyes, such as trypan blue.

Reduced cell lysis also may be evidenced by the decrease in contaminating cellular proteins that are released from cells upon cell lysis. In some aspects, the contaminating cellular proteins are cellular proteins from white blood cells, red blood cells, and/or platelets (when the analytes of the proteomic analysis is not proteins of white blood cells, red blood cells, and/or platelets). In some aspects, the composition described herein reduces cell lysis of white blood cells, red blood cells, and/or platelets so that there is a reduced level of contaminating cellular proteins from these cells. While a minimal or base level of contaminating cellular proteins may be present, due to the shear of collecting the blood from the subject, for example, the amount of contaminating cellular proteins increases over time, e.g., upon storage at refrigerated temperatures or higher temperatures. As a result of the composition imparting stability, the protein sample yielded in the method is suitable for proteomic analysis due to the reduced level in contaminating cellular proteins. In some aspects, the reduced level in contaminating cellular proteins is a reduction of at least about 10% (e.g., at least about 20%, at least about 30%, at least about 40%, at least about 50%, at least about 60%, at least about 70%, at least about 80% at least about 90%, or more) relative to the level of contaminating cellular proteins of a control protein sample obtained from an isolated fraction of a blood sample collected in a blood collection tube without the composition described herein (e.g., comprising (1) citrate-based anticoagulant (AC), an aldehyde releaser (AR), a calcium channel blocker, and a sodium channel blocker or (2) a citrate-based anticoagulant (AC), an aldehyde releaser (AR), and a sodium channel blocker). In some aspects, the reduced level in contaminating cellular proteins is a reduction of at least about 10% (e.g., at least about 20%, at least about 30%, at least about 40%, at least about 50%, at least about 60%, at least about 70%, at least about 80% at least about 90%, or more) relative to the level of contaminating cellular proteins of a control protein sample obtained from an isolated fraction of a blood sample collected in a blood collection tube without the composition described herein, following storage of the mixture for at least 48 hours prior to the step of isolating the fraction or cellular fraction (optionally, for at least 48 hours but less than 7 days prior to the isolating step, or optionally for at least 48 hours but less than 14 days prior to the isolating step), wherein the storage is at a temperature greater than 4° C., optionally, at a temperature of about 20° C. to about 25° C. Methods of measuring contaminating cellular proteins are known in the art and include for example, measurement of a representative contaminating cellular protein. In some aspects, the representative contaminating cellular protein is a protein of the red blood cell proteome, described in Pasini et al., Blood 108 (3): 791-801 (2006) or Bryk and Wisniewski, J Proteome Res 16:2752-2761 (2017). In some aspects, the representative contaminating cellular protein is hemoglobin, or a subunit thereof (HbA, HbB, HbD, HbG, HbZ), or carbonic anhydrase (CA1), or a peroxiredoxin (e.g., PRDZ1, PRDX12, PRDX16), biliverdin reductase B (BLVRB), catalase (CAT), superoxide dismutase (SOD1), bisphosphoglycerate mutase (BPGM). In some aspects, the representative contaminating cellular protein is a protein of the white blood cell proteome, e.g., leukocyte-specific protein 1 (LSP1), a subunit of the T-cell receptor, a subunit of the B-cell receptor. In some aspects, the representative contaminating cellular protein is a protein of the platelet proteome, such as those described in Senzel et al., Curr Opin Hematol 16 (5): 329-333 (2009) and Doyle et al., Blood J 55 (1): 82-84. In some aspects, the representative contaminating cellular protein is beta-thromboglobulin, and platelet factor 4.

Levels of cell lysis may also be measured by measuring cell stabilization, as represented by cell-free DNA using TapeStation, BioAnalyzer, PCR, droplet digital PCT (ddPCR), or digital PCR. See, e.g., Norton et al., Clin Biochem 46:1561-1565 (2013).

In various aspects, the low levels of cell lysis may be evident from the slope of the best fit line of a line graph of the number of proteins in the protein sample yielded from step (b) plotted as a function of storage time. In exemplary aspects, the slope of the best fit line of a line graph of the number of proteins in the protein sample yielded from step (b) plotted as a function of storage time is closer to 0 compared to the slope of the best fit line of a line graph of the number of proteins in a control blood sample not contacted with a composition (e.g., comprising citrate-based anticoagulant (AC), an aldehyde releaser (AR), a calcium channel blocker, and a sodium channel blocker).

In various aspects, the low levels of cell lysis may be evident from the slope of the best fit line of a line graph of the number of peptides in the protein sample yielded from step (b) plotted as a function of storage time. In exemplary aspects, the slope of the best fit line of a line graph of the number of peptides in the protein sample yielded from step (b) plotted as a function of storage time is closer to 0 compared to the slope of the best fit line of a line graph of the number of peptides in a control blood sample not contacted with a composition.

Plasma Fractions and Increased Levels of Low Abundance Plasma Proteins or Unique Peptides per Protein or Unique Proteins Identified Upon Storage

In some aspects, the fraction isolated from the mixture is a plasma fraction and the protein sample yielded in the method is suitable for proteomic analysis due to an increased level of low-abundance plasma proteins. In some aspects, the increased level of low-abundance plasma proteins is an increase of at least about 10% (e.g., at least about 20%, at least about 30%, at least about 40%, at least about 50%, at least about 60%, at least about 70%, at least about 80% at least about 90%, or more) relative to the level of low-abundance plasma proteins present in a control protein sample obtained from an isolated fraction of a blood sample collected in a blood collection tube without a composition (e.g., comprising (1) a citrate-based anticoagulant (AC), an aldehyde releaser (AR), a calcium channel blocker, and a sodium channel blocker, or (2) a citrate-based anticoagulant (AC), an aldehyde releaser (AR), and a sodium channel blocker). In some aspects, the increased level of low-abundance plasma proteins is an increase of at least about 10% (e.g., at least about 20%, at least about 30%, at least about 40%, at least about 50%, at least about 60%, at least about 70%, at least about 80% at least about 90%, or more) relative to the level of low-abundance plasma proteins present in a control protein sample obtained from an isolated fraction of a blood sample collected in a blood collection tube without a composition (e.g., comprising (1) a citrate-based anticoagulant (AC), an aldehyde releaser (AR), a calcium channel blocker, and a sodium channel blocker or (2) a citrate-based anticoagulant (AC), an aldehyde releaser (AR), and a sodium channel blocker), following storage of the mixture for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring levels of low-abundance plasma proteins are known in the art. See e.g., Liu et al., PLOS One DOI: 10.1371/journal.pone.0166306 (2016), which also describes over 125 low-abundance plasma proteins.

In some instances, the fraction isolated from the mixture is a plasma fraction and the protein sample yielded in the method is suitable for proteomic analysis due to an increased level of unique peptides identified per protein. In exemplary instances, the unique peptides identified per protein are determined by discovery-label-free data dependent acquisition (DDA) LC-MS/MS. In some aspects, the increased level of unique peptides identified per protein is an increase of at least about 10% (e.g., at least about 20%, at least about 30%, at least about 40%, at least about 50%, at least about 60%, at least about 70%, at least about 80% at least about 90%, or more) relative to the level of unique peptides identified per protein present in a control protein sample obtained from an isolated fraction of a blood sample collected in a blood collection tube without a composition (e.g., comprising citrate-based anticoagulant (AC), an aldehyde releaser (AR), a calcium channel blocker, and a sodium channel blocker). In some aspects, the increased level of unique peptides identified per protein is an increase of at least about 10% (e.g., at least about 20%, at least about 30%, at least about 40%, at least about 50%, at least about 60%, at least about 70%, at least about 80% at least about 90%, or more) relative to the level of unique peptides identified per protein present in a control protein sample obtained from an isolated fraction of a blood sample collected in a blood collection tube without a composition (e.g., comprising citrate-based anticoagulant (AC), an aldehyde releaser (AR), a calcium channel blocker, and a sodium channel blocker), following storage of the mixture for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. Methods of measuring the level of unique peptides identified per protein may be carried out by DDA LC-MS/MS as essentially described in Almazi et al., Proteomics Clin Applications 12: 1700121 (2018); doi: 10.1002/prca.201700121.

In some instances, the fraction isolated from the mixture is a plasma fraction and the protein sample yielded in the method is suitable for analysis due to an increased level of unique proteins identified, as determined by LC-MS/MS, optionally, wherein the unique proteins are secretory proteins. In some aspects, the increased level of unique proteins identified is an increase of at least about 10% (e.g., at least about 20%, at least about 30%, at least about 40%, at least about 50%, at least about 60%, at least about 70%, at least about 80% at least about 90%, or more) relative to the level of unique proteins identified present in a control protein sample obtained from an isolated fraction of a blood sample collected in a blood collection tube without a composition (e.g., comprising (1) a citrate-based anticoagulant (AC), an aldehyde releaser (AR), a calcium channel blocker, and a sodium channel blocker or (2) a citrate-based anticoagulant (AC), an aldehyde releaser (AR), and a sodium channel blocker). In some aspects, the increased level of unique proteins identified is an increase of at least about 10% (e.g., at least about 20%, at least about 30%, at least about 40%, at least about 50%, at least about 60%, at least about 70%, at least about 80% at least about 90%, or more) relative to the level of unique proteins identified present in a control protein sample obtained from an isolated fraction of a blood sample collected in a blood collection tube without a composition (e.g., comprising (1) a citrate-based anticoagulant (AC), an aldehyde releaser (AR), a calcium channel blocker, and a sodium channel blocker or (2) a citrate-based anticoagulant (AC), an aldehyde releaser (AR), and a sodium channel blocker), following storage of the mixture for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. Methods of measuring the level of unique proteins identified may be carried out by LC-MS/MS as essentially described in Tsung-Heng Tsai et al., Proteomics 15(13): 2369-2381 (2015) and Geyer et al., Cell Systems 2:185-195 (2016).

### Likeness to Freshly Isolated Blood Sample

In some aspects, the blood sample stored in a composition described herein is suitable for subsequent analysis due its likeness to a freshly isolated blood sample, in terms of intact protein/nucleic acid content, even after the sample has been stored. As used herein, the term “freshly isolated” in “freshly isolated blood sample” refers to a blood sample wherein not more than 26 hours has passed since the time the blood sample was isolated, collected or drawn from a subject. In various instances, the sample comprises greater than about 50%, greater than about 60% (e.g., greater than about 70%, greater than about 80%, greater than about 90% or more) of the intact proteins present in a freshly isolated blood sample. In various instances, blood sample comprises greater than about 50%, greater than about 60% (e.g., greater than about 70%, greater than about 80%, greater than about 90% or more) of the intact proteins present in a freshly isolated blood sample, even following storage of the sample for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring intact protein content include, for example, SDS-PAGE, immunoassay, and mass spectrometry.

Also, in various instances, the likeness to a freshly isolated blood sample may be evident from the number of plasma proteins and/or peptides present in the protein sample following storage for at least 48 hours being very similar to the number of plasma proteins and/or peptides present in the protein sample within about 0 hours to about 4 hours of collecting the blood sample from a subject. In various instances, the number of plasma proteins and/or peptides present in the sample following storage for at least 48 hours is within about 20% or about 25% of the number of plasma proteins and/or peptides present in the sample within about 0 hours to about 4 hours of collecting the blood sample from a subject. In various instances, the number of plasma proteins and/or peptides present in the sample following storage for at least 48 hours is within about 10% of the number of plasma proteins and/or peptides present in the sample within about 0 hours to about 4 hours of collecting the blood sample from a subject. In various instances, the number of plasma proteins and/or peptides present in the sample following storage for at least 48 hours is within about 7.5% or about 5% of the number of plasma proteins and/or peptides present in the sample within about 0 hours to about 4 hours of collecting the blood sample from a subject.

### Reduced Contaminating Protein Products

In some aspects, the composition allows for a reduced level of contaminating protein products in a blood sample, relative to the level of contaminating protein products in a control sample obtained from an isolated fraction of a blood sample collected in a blood collection device without the composition described herein (e.g., comprising (1) a citrate-based anticoagulant (AC), an aldehyde releaser (AR), a calcium channel blocker, and a sodium channel blocker or (2) a citrate-based anticoagulant (AC), an aldehyde releaser (AR), and a sodium channel blocker), following storage of the blood sample or control sample derived for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. In exemplary instances, the protein sample yielded from the blood sample comprises less than about 40%, (e.g., less than about 35%, less than about 30%, less than about 25%, less than about 20%, less than about 15%, less than about 10%, or less than about 5%) of the contaminating protein products, relative to the level of contaminating protein products in a control blood sample obtained from an isolated fraction of a blood sample collected in a blood collection device without the composition described herein (e.g., comprising (1) citrate-based anticoagulant (AC), an aldehyde releaser (AR), a calcium channel blocker, and a sodium channel blocker or (2) a citrate-based anticoagulant (AC), an aldehyde releaser (AR), and a sodium channel blocker), following storage of the blood sample from which the protein sample or control sample derived for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. As used herein “contaminating protein products” refer to oxidized, reduced, amidated, deamidated, lysed, degraded, aggregated, and/or precipitated protein products. Methods of measuring contaminating protein products are known in the art and include for example HPLC and MS.

The following examples are given merely to illustrate the present invention, and the advantages thereof, and not in any way to limit its scope.

### EXAMPLES

### Example 1—First Round Screening of Platelet Activation Inhibitors

Platelet activation inhibitors (e.g., tetracaine (2 mM), mexiletine (100 μM, 500 μM, 2 mM), verapamil (40 μM, 200 μM, 1 mM), quinidine (20 μM, 100 μM, 500 μM), benzocaine (100 μM), aspirin (20 μM, 100 μM, 500 μM), carbamazepine (50 μM), ticlopidine (20 μM, 100 M, 500 μM), procaine (4 mM, 8 mM, 16 mM), doxycycline (20 M, 100 μM), diltiazem (20 μM, 100 μM, 500 M), bupivacaine (100 μM, 500 μM, 2 mM)) were added to Streck B formulation (i.e., IDU, ACD-A, and α-cyclodextrin) at various concentrations, then prefilled to blood collection tubes for venous blood draws. Blood specimens drawn from 3 donors were stored at room temperature for up to 5 days. Activated platelet secret granule contents, such as growth factors, chemokines, and cytokines, into plasma. Elevated plasma concentration of TGF-β1 indicates platelet activation and granule release. Plasma TGF-β1 level was measured with immunoassay using Ella Automated Immunoassay System. Freshly drawn blood (day 0), blood stored for 1 day (Day 1), 3 days (Day 3), and 7 days (Day 7) were tested.

The tested formulations are compared with EDTA and ACD-A for their ability to prevent granule release from platelets. Streck B formulation spiked with 2 mM tetracaine was also used as a reference. If a formulation containing a tested inhibitor behaved comparable, if not better than Streck B formulation with tetracaine in maintaining TGF-1, the inhibitor is considered as a lead chemical. Of the twelve platelet activation inhibitors tested, eight (i.e., diltiazem, mexiletine, quinidine, bupivacaine, doxycycline, lidocaine, mepivacaine, or verapamil) graduated from the first round of the screening (data not shown).

### Example 2—Mexiletine and Verapamil Identified as Top Platelet Activation Inhibitor Candidates

During the second round of screening, eight platelet activation inhibitors that graduated from the first round of screening (i.e., diltiazem, mexiletine, quinidine, bupivacaine, doxycycline, lidocaine, mepivacaine, or verapamil) were tested at 3 to 5 different dosages to evaluate their ability to reduce platelets activation and to identify proper working concentration. Preservative formulations were made by adding platelet activation inhibitors to the Streck B formulation at the desired concentrations. Venous blood specimens from 2 donors were then drawn into blood collection tubes (BCTs) prefilled with these formulations. As references, blood specimens were also drawn into EDTA tubes, ACD-A tubes, tubes filed with Streck B formulation, and tubes containing Streck B formulation spiked with and 2 mM tetracaine. Blood specimens were stored at room temperature for up to 7 days.

The percentages of activated platelets (CD62P positive) among total platelets (CD61 positive) in drawn whole blood were measured by flow cytometry. The percentage of activated platelets was tested with Day 0 and Day 3 blood specimens. The plasma levels of TGF-β1 in Day 0, Day 1, Day 3, and Day 7 old blood specimens were measured with immunoassay.

Doxycycline appeared to be light-sensitive and was determined to be unsuitable for commercial use. The remaining seven platelet activation inhibitors (i.e., diltiazem, mexiletine, quinidine, bupivacaine, lidocaine, mepivacaine, or verapamil) at the tested concentrations behaved at least comparable to each other (data not shown).

The effects of these seven inhibitors on white blood cells, red blood cells, and platelets were evaluated during the third round of screening. If an inhibitor has a detrimental effect on blood cells or cell fragments, cellular contents will be released into the plasma. Therefore, plasma levels of myeloperoxidase (MPO), ferritin, and RANTES were measured with immunoassay as damage indicators of white blood cells, red blood cells, and platelets, respectively.

Preservative formulations were made by adding platelet activation inhibitors to Streck B formulation at concentrations determined through the second round of screening. Venous blood was collected from 2 donors for all preservative formulations. EDTA, ACD-A, Streck B formulation and Streck B formulation spiked with 2 mM Tetracaine were used as references. Draw whole blood samples were stored at room temperature for up to 7 days.

Lidocaine, mepivacaine, verapamil, mexiletine, diltiazem, bupivacaine, and quinidine showed little adverse effects on white blood cells, red blood cells and platelet throughout the 7-day room temperature storage (data now shown). The seven tested platelet activation inhibitors behaved similarly to tetracaine. Results showed that formulations containing a platelet activation inhibitor had significantly lower plasma levels of RANTES compared to EDTA, ACD-A, and Streck B formulation throughout the 7-day storage, indicating the ability of chemicals to prevent platelets from activation (data not shown). Mepivacaine was dropped from the further testing due to being cost prohibitive.

### Example 3—Functional Stability of Ion Channel Inhibitors

Six lead platelet activation inhibitors (i.e., (i.e., diltiazem, mexiletine, quinidine, bupivacaine, lidocaine, or verapamil) along with tetracaine were tested for their functional stability. The preservative formulations were made by adding one of the six platelet activation inhibitors to Streck B formulation. Blood collection tubes filled with preservative formulations were stored at 2° C., 22° C., 30° C., 37° C. and 50° C. for up to 8 weeks. EDTA, ACD-A, and tubes pre-filled with Streck B formulation were also aged in the same manner and serves as references.

Freshly made tubes, 2 weeks old tubes, 4 weeks old tubes, and 8 weeks old tubes were then used to collect venous blood from 2 self-proclaimed healthy donors. Blood specimens were stored at room temperature for up to 3 days. The percentage of platelet activation and plasma level of TGF-31 to test the ability of the aged formulation to prevent platelets activation at collection time and during room temperature storage. Aged formulations containing 2 mM of mexiletine or 1 mM of verapamil demonstrated better functional stability compared to that of other tested ion inhibitors (data not shown).

### Example 4—Combination of Mexiletine and Verapamil

The study aimed to test if combining mexiletine and verapamil would improve the functionality of the preservative formulations. Formulations with a fixed concentration of verapamil and various concentrations of mexiletine were tested. For instance, Streck MO formulation (M100%) comprises citric acid (12 g/L to 17 g/L), trisodium citrate (60 g/L to 70 g/L), dextrose (68 g/L to 78 g/L), imidazolidinyl urea (140 g/L to 160 g/L), verapamil (3 g/L to 5 g/L), and mexiletine (3 g/L to 5 g/L). Streck MH formulation (M0%) comprises comprises citric acid (12 g/L to 17 g/L), trisodium citrate (60 g/L to 70 g/L), dextrose (68 g/L to 78 g/L), imidazolidinyl urea (140 g/L to 160 g/L), verapamil (3 g/L to 5 g/L), but no mexiletine. These formulations are compared with EDTA, ACD-A, Streck B formulation, and Streck MH formulation for their ability to minimize platelet activation.

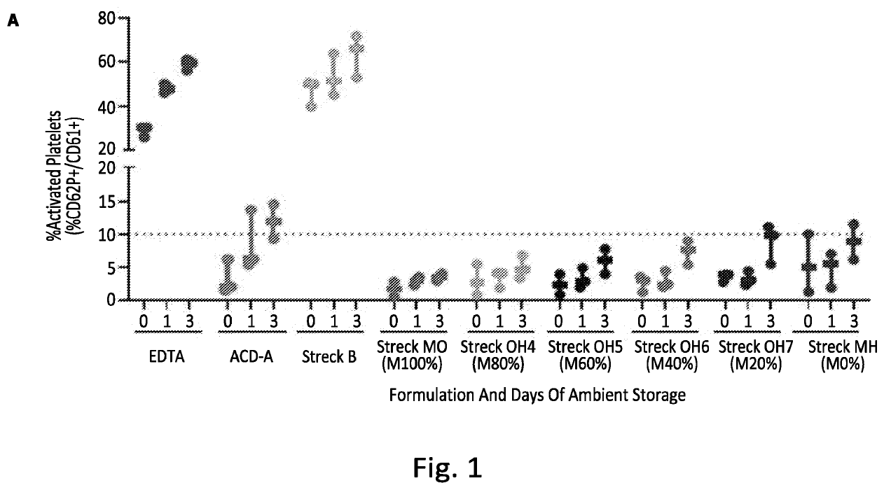

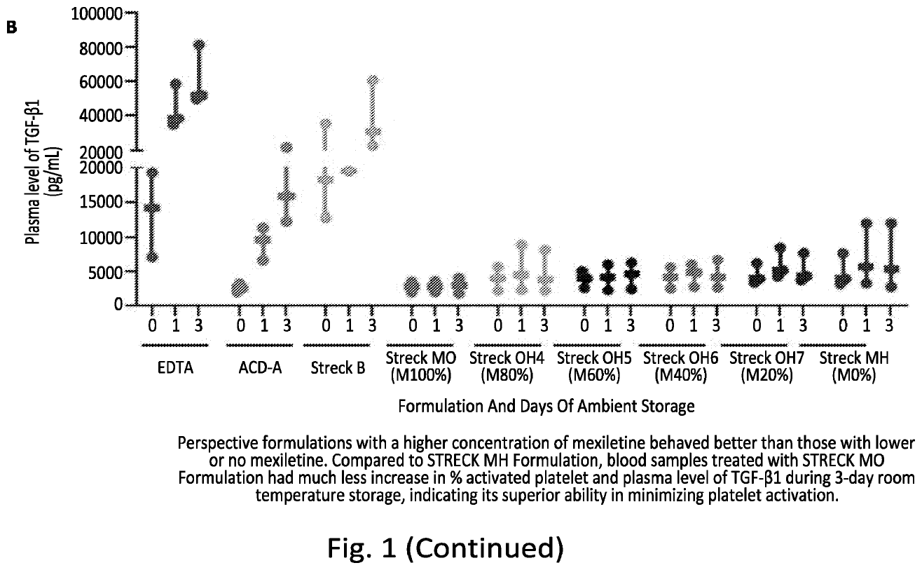

Venous blood from three self-proclaimed healthy donors was drawn into BCTs prefilled with the formulations and stored at room temperature. The percentage of activated platelets was measured by flow cytometry. The plasma level of TGF-β1 was with immunoassay as a marker of granule release from activated platelets. Functionally compared with the Streck MH formulation, Streck MO formulation showed superior ability in minimizing platelet activation and subsequent granule release during 3 days of room temperature storage (FIG. 1). Results showed that blood collected with Streck MO formulation had little increase in % activation platelets and plasma level of TGF-β1, while blood collected with Streck MH had modest but significant increase in platelet granule release during ambient storage. Thus, mexiletine is crucial for the ability of the Streck MO formulation to stabilize blood analytes.

### Example 5—Venous Blood Sample Data

### Protein Markers:

### a). Venous Blood Samples Tested Via Ella Simple Plex Assays (Immunoassay):

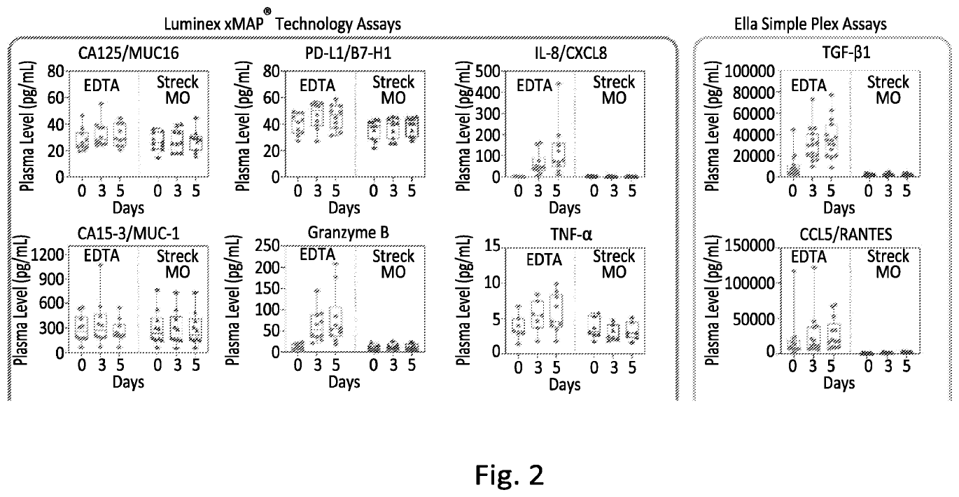

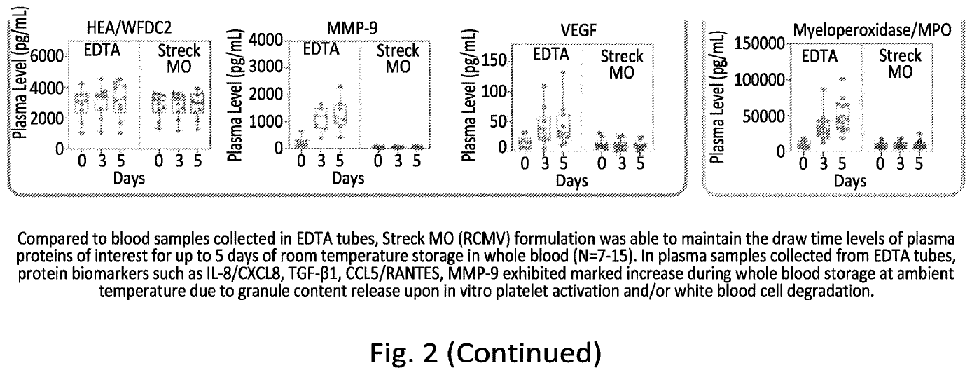

Blood samples from 15 self-proclaimed healthy donors were drawn into evacuated blood collection tubes containing Streck MO Formulation or EDTA tubes and stored at room temperature for up to 5 days. Plasma was isolated using a general double-spin protocol (1800×g for 15 min, followed by 2800×g for 15 min, both at room temperature), aliquoted and frozen at −80° C. until use. Functionally compared with EDTA and ACD-A, the Streck MO formulation maintained draw-time levels of plasma proteins of interested, inducing TGF-β1, RANTES, and myeloperoxidase for up to 5 days of room temperature storage as measured by immunoassay (FIG. 2). In addition, Streck MO formulation appeared to have no adverse effect on white blood cells.

b) Venous Blood Samples Tested with Luminex xMAP Multiplex Immunoassay Panel (R&D System):

Blood from 10 self-proclaimed healthy donors was drawn tubes prefilled with Streck MO Formulation and stored at room temperature. After double-spin process, isolated plasma fractions were tested with Luminex xMAP Multiplex Immunoassay Panel.

Functionally compared with EDTA, Streck MO Formulation maintained draw-time levels of 50 out of 55 tested plasma proteins (see Table 1 below).

TABLE 1
Luminex Multiplex Immunoassay Study 2

| Analytes that are relatively stable | Analytes that are NOT stable in EDTA or ACD-A, but are | | Analytes that are not stable |
|---|---|---|---|
| in all tested BCTs (15/55) | relatively stable in Streck MO formulation (35/55) | | in any tested BCT (5/55) |
| CA15- [symbol] /MUC-1 | Angiopo [symbol] etin-1 | IL-1beta/IL- [symbol] F2 | CD [symbol] /TNFR [symbol] F5 |
| CA125/MUC16 | BDNF | IL-1 [symbol] /IL- [symbol] F3 | CXCL10/IP-10/CRG [symbol] 2 |
| C [symbol] ACAM-5/CD66e | CCL2/ [symbol] E/MCP-2 | IL-7 | [symbol] GF basic/FGF [symbol] /b [symbol] GF |
| CD27/TNFR [symbol] F7 | CCLS/RANTES | IL- [symbol] | [symbol] nsulin |
| CXCL [symbol] /GR [symbol] alpha/KC/CINC-1 [symbol] | CD40 Ligand/TNF [symbol] F5 | MICA | Prolactin |
| [symbol] | Complement Component C9 | MIF | |
| GITR/TNFRSF18 | CXCL2/GRO beta/MIP-2/CINC-3 | MMP-1 | |
| HE4/WFDC2 | CXCL5/ENA-78 | MMP- [symbol] | |
| IL-15 | CXCL6/GCP-2 | Myeloperoxidase/MPO | |
| Leptin/OB | EGF | [symbol] -Selectin/CD52P | |
| MMP- [symbol] | Fas Ligand/TNFSF6 | PDGF- [symbol] | |
| PD-L1/ [symbol] 7-H1 | G-CSF | PDGF- [symbol] | |
| Osteopontin/CPN | Galectin-1 | TGF-alpha | |
| | | ( [symbol] ) | |
| SC [symbol] Ligand | Galectin-3 | TNF-alpha | |
| TRAIL/TNF [symbol] F10 | Galectin-9 | TREM-1 | |
| | Granzyme A | VEGF | |
| | Granzyme B | VEGF-C | |
| | HGF | | |

(* Analytes < LLOQ are excluded) [symbol]
indicates data missing or illegible when filed

The plasma concentrations of representative proteins and cytokines are plotted in FIG. 2. Streck MO Formulation maintained the draw time concentration of all plotted protein markers during 5-day room temperature storage. In contrast, EDTA-anticoagulated blood showed elevated levels of MPO, TGF-β1, CCL5/RANTES, MMP-9, VEGF, Granzyme B, and TNFα after being stored at room temperature for 3 days. Thus, it indicates the ability of Streck MO formulation to stabilize protein/cytokine in drawn whole blood during storage.

### B. In Vitro Hemolysis:

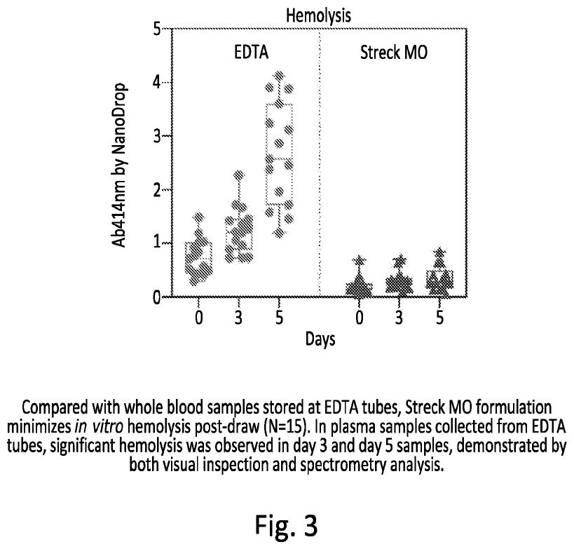

Venous blood from 15 self-proclaimed healthy donors was drawn into EDTA tubes and blood collection tubes containing Streck MO Formulation, then stored at room temperature for up to 5 days. Hemolysis was quantitatively measured by following the absorbance at 414 nm with Nanodrop. An increased A414 indicates releasing of hemoglobin from broken RBC, which is a key event of hemolysis. EDTA-anticoagulated blood had in vitro hemolysis since day 3 (FIG. 3). Streck MO Formulation minimized in vitro hemolysis for up to 5 days at room temperature.

### C. Flow Cytometry:

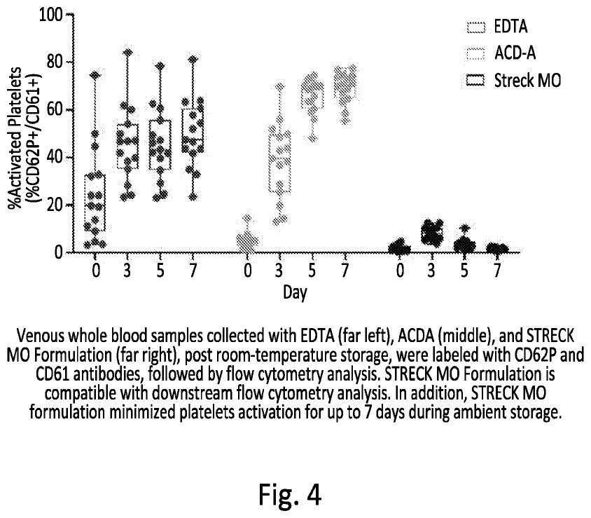

Venous Blood from 15 self-proclaimed healthy donors was drawn into EDTA tubes, ACD-A tubes, tubes pre-filled with Streck MO Formulation. Drawn blood samples were stored at room temperature for up to 7 days, then labeled with CD62P and CD61 antibodies and analyzed by flow cytometry. Streck MO Formulation is compatible with flow cytometry analysis for cell surface antigens. Functionally compared with EDTA and ACD-A, STRECK MO Formulations were shown to minimize platelet activation for up to 7 days during room temperature storage (FIG. 4).

D. cfDNA Stability:

Venous Blood from 5 self-proclaimed healthy donors was drawn into EDTA tubes or evacuated blood collection tubes containing Streck MO Formulation. The drawn blood samples were stored at room temperature for up to 7 days. Total cell-free nucleic acids were isolated from day 0 or day 7 blood samples using Qiagen's QIAamp Circulating Nucleic Acid Kit.

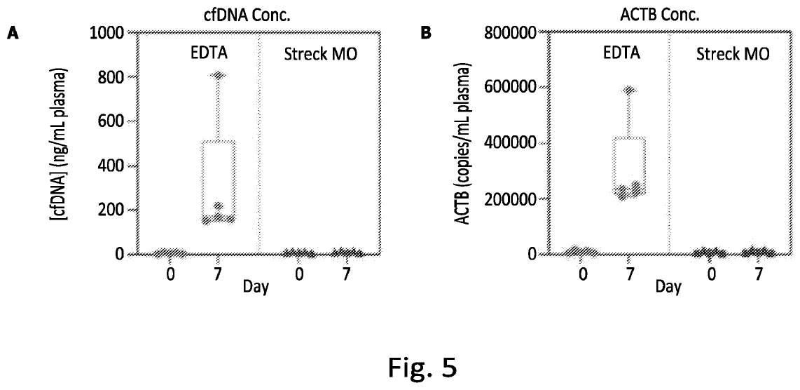

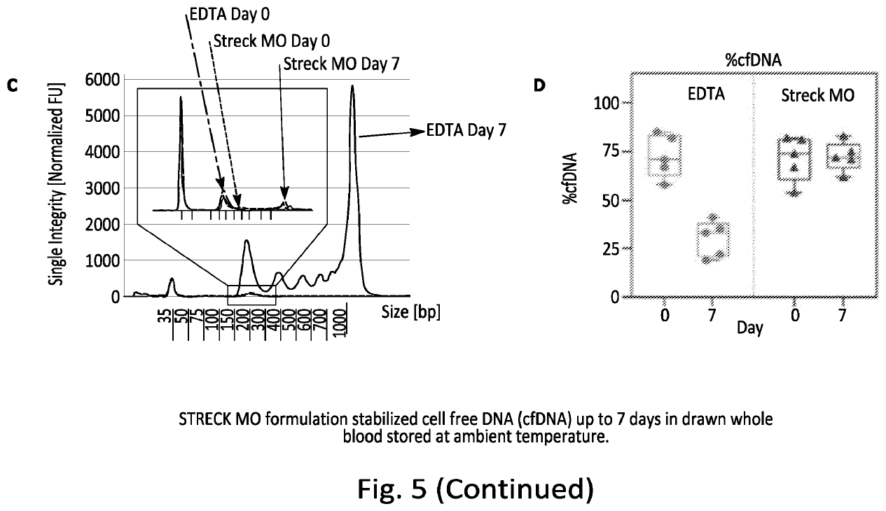

The resultant plasma cfDNA was first studied with Qubit HS DNA Assay (FIG. 5A), which quantifies the concentration of total plasma cfDNA. Without proper stabilization, white blood cells in drawn blood will lyse during storage and release large amounts of genomic DNA fragments into the plasma, leading to elevated total plasma cfDNA. Compared to cfDNA originating from white blood cells, tumor-derived cfDNA is preset at a very low concentration. Tumor-derived cfDNA should not be detectable in healthy donors. Thus, plasma from day 0 EDTA blood, day 0 Streck MO blood, and day 7 Streck MO blood all had low concentrations of cfDNA, suggesting these samples had little genomic DNA contamination. However, plasma from day 7 EDTA blood had elevated total cfDNA, indicating the plasma contained cfDNA of white-blood-cell origin. Consistent with this data, examining the plasma level of housekeeping gene β-actin (ACTB) with digital droplet PCR also found plasma cfDNA from day 7 EDTA blood contains a high amount of ACTB (FIG. 5B), indicating genomic DNA contamination. On the other hand, Streck MO maintained plasma ACTB at the drawn level after 7 days of ambient storage.

Plasma cfDNA was further analyzed with Agilent cfDNA ScreenTape Assay on Agilent TapeStation (FIGS. 5C & 5D). Plasma cfDNA isolated from Fresh (Day 0) and Day 7 Streck MO blood shared electropherogram plots similar to that of fresh EDTA blood. On the contrary, electropherogram plots of the plasma cfDNA isolated from day 7 EDTA blood contained a prominent peak of apoptotic-ladder DNA, indicating plasma cfDNA was contaminated with white blood cell genomic DNA (FIG. 5 C). The boxed area in FIG. 5C highlighted a population of cfDNA between 150 to 300 bp. Tumor-derived cfDNA was previously reported to be found in this cfDNA. Thus, the percentage of cfDNA between 150-300 bp among total cfDNA (% cfDNA) was used to indicate genomic DNA contamination (FIG. 5D). Fresh blood sample with little lysed white blood cells has a lower chance of genomic DNA contamination, therefore, has a higher % cfDNA. Day 0 EDTA blood, Day 0 Streck MO blood, and Day 7 Streck MO blood all had a % cfDNA level of 75%. However, Day 7 EDTA only had 30% of % cfDNA, suggesting that total cfDNA in aged EDTA plasma contains a higher amount of white blood cell-derived cfDNA. Results demonstrated that Streck MO Formulation maintained the concentration and population of cfDNA during the 7-day ambient storage of drawn whole blood.

E. cfRNA Stability:

Venous Blood from 5 self-proclaimed healthy donors was drawn into EDTA tubes or evacuated blood collection tubes containing STRECK MO formulation and stored at room temperature for up to 7 days. Total cell-free nucleic acids were isolated from day 0 or day 7 blood samples using Qiagen's QIAamp Circulating Nucleic Acid Kit.

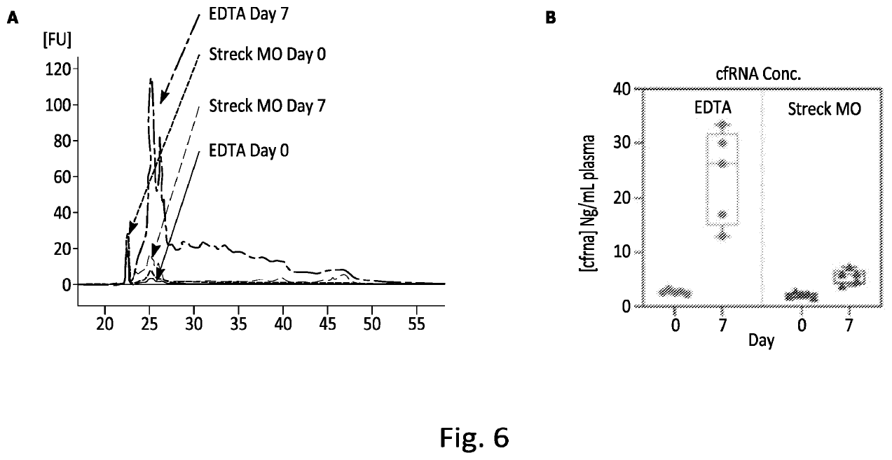

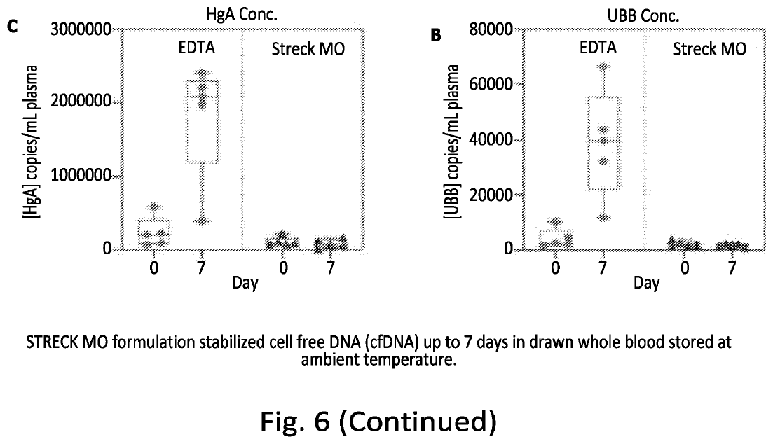

The plasma cfRNA was first analyzed with Agilent's RNA Pico Kit on the Agilent Bioanalyzer system (FIG. 6A). Even after 7 days of ambient storage, cfRNA isolated from STRECK MO blood had roughly the same electropherogram plots as that of freshly collected blood (STRECK MO and EDTA). In contrast, both the concentration and the population of cfRNA were greatly changed in day 7 EDTA blood versus day 0 EDTA blood.

Qubit miRNA Assay was used to quantify the concentration of total plasma cfRNA. If RNA-containing cells, such as reticulocytes and white blood cells, were lysed during storage, then large amounts of RNA fragments would be released into plasma, masking the very low abundant circulating cfRNA while resulting in elevated cfRNA. Compared to plasma from day 0 Streck MO blood, plasma from day 7 Streck MO blood only had slightly increased total cfRNA concentration (FIG. 6B). The increase was nearly 5-fold higher in EDTA blood after 7 days of ambient storage.

Digital droplet PCR was used to quantify the plasma concentrations of Hemoglobin A mRNA (HgA) and ubiquitin B mRNA (UBB). HgA is a red blood cell-specific gene while UBB is a housekeeping gene found in both red blood cells and white blood cells. Streck MO formulation maintained the draw time plasma levels of HgA (FIG. 6C) and UBB (FIG. 6D) Venous Blood from 5 self-proclaimed healthy donors was drawn into blood collection tubes prefilled with Streck MO formulation and stored at room temperature. Results demonstrated that the Streck MO formulation maintained draw-time levels of cfDNA for up to 7 days as measured by Qubit High Sensitivity DNA analysis, cfDNA Screen tape analysis.

### F. Formulation Stability (HPLC-Based Chemical Stability):

The Streck MO formulation currently has a closed vial shelf life of 9 months at 2° C. to 30° C.

### Example 6—Capillary Blood Sample Data

A. Capillary Blood Samples Collected with Yourbio's TAP II Devices:

The purpose of the study was to test the feasibility of applying the Streck MO formulation using a TAP II microcollection device. TAP II is a single-use device that collects up to 0.25 mL of capillary blood from a donor's upper arm. TAP II device can house a RAM Scientific's capillary blood collection tube. Spray-coated EDTA tubes were used for this study. Due to the lack of RAM tubes spray-coated with Streck MO formulation, Streck MO formulation was spiked into EDTA anticoagulated blood post-TAP II collection.

Capillary blood samples were collected from 10 donors, using four EDTA-TAP II devices per donor (4 tubes of blood per donor). Two tubes of EDTA blood per donor were spiked in with Streck MO formulation immediately after collection. As a reference, venous blood samples from the same 10 donors were collected using EDTA tubes and tubes prefilled with Streck MO formulation. Blood samples were stored at room temperature for up to 3 days.

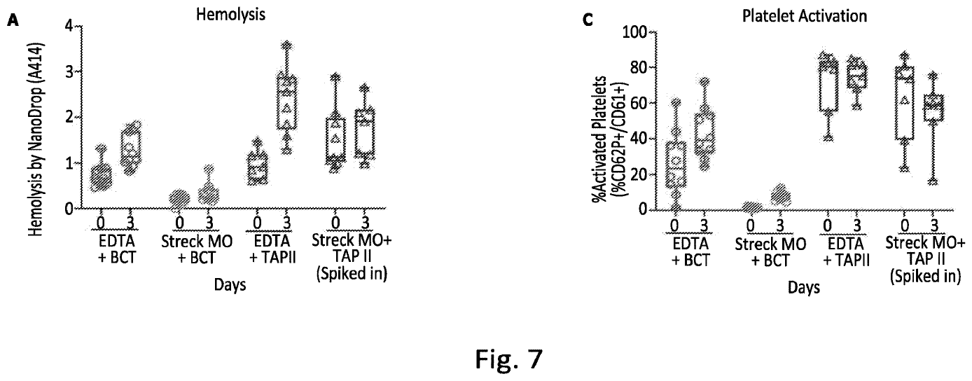

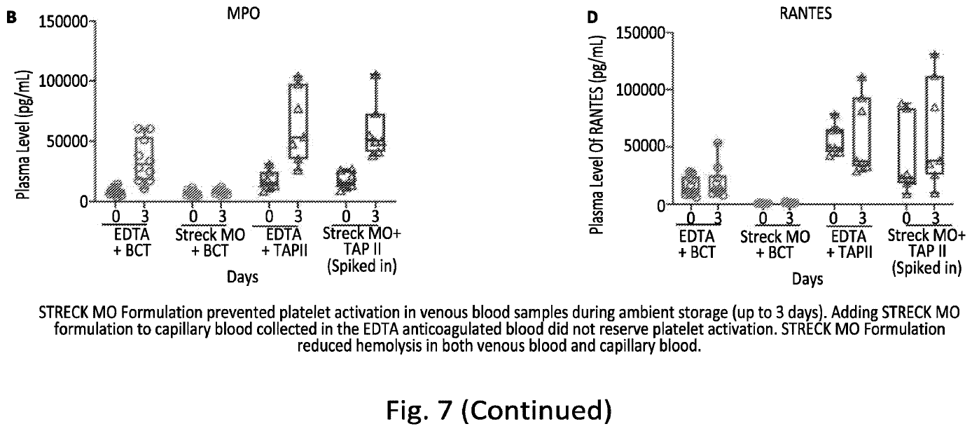

Four functionality assays were conducted, including measuring hemolysis by detecting absorbance at 414 nm (FIG. 7A), determining percentage of activated platelets using flow cytometry (FIG. 7C), and measuring the level of white blood cell marker MPO (FIG. 7B) and platelet activation marker RANTES (FIG. 7D) with immunoassay.

Capillary blood samples collected via TAP II had a higher level of in vitro hemolysis compared to venous blood samples. In both EDTA venous blood and EDTA capillary blood, room-temperature storage induces hemolysis. Streck MO formulation minimized the hemolysis for up to 3 days in both venous blood and capillary blood.

Results demonstrated that Streck MO formulation prevented platelets activation in venous blood samples and stabilized plasma MPO and RANTES concentration for up to 3 days during room temperature storage. Adding Streck MO formulation to capillary blood collected in EDTA tubes did not reverse platelet activation or minimize granule release during room temperature storage.

B. Capillary Blood Samples Collected with Tasso's Tasso+ Devices:

The feasibility of applying Streck MO formulation using a Tasso+ device was tested. Tasso+ is a single-use device that collects up to 0.5 mL of capillary blood from a donor's upper arm. Tasso+ is compatible with BD Microtainer® Blood Collection Tubes. Spray-coated EDTA microtainer tubes and empty microtainer tubes filled with 25 μL of Streck MO formulation were used for collecting capillary blood.

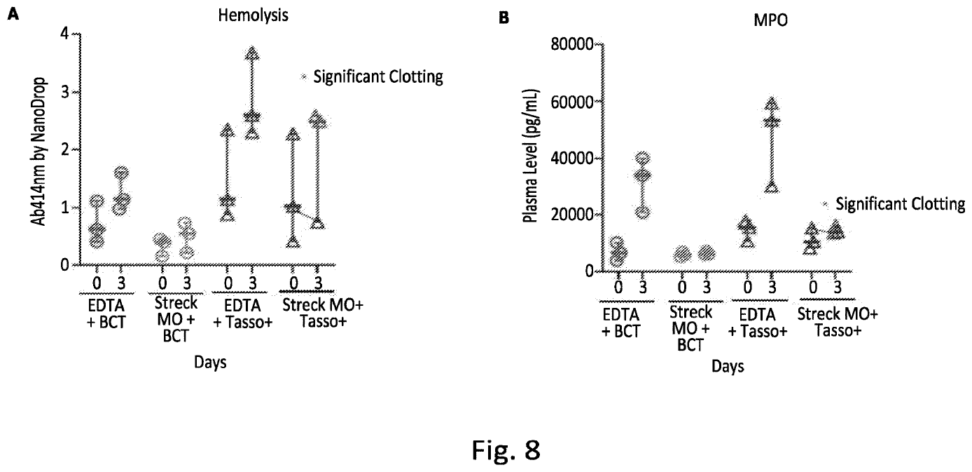

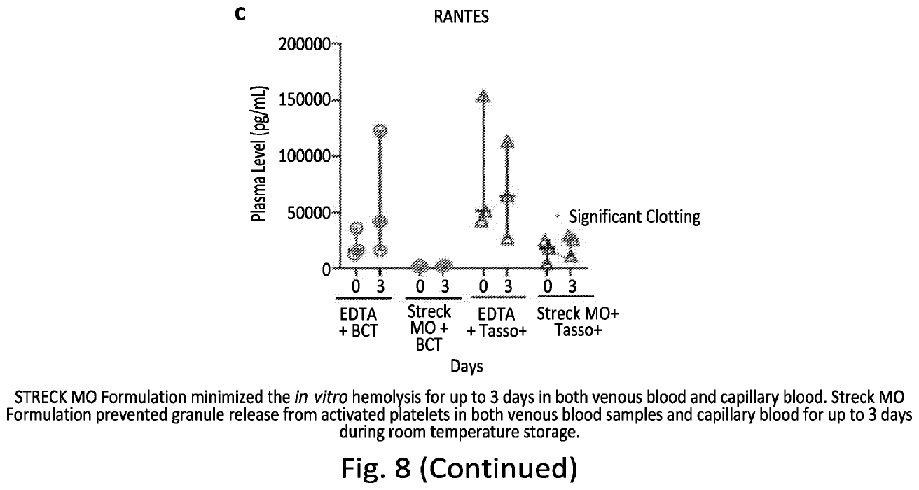

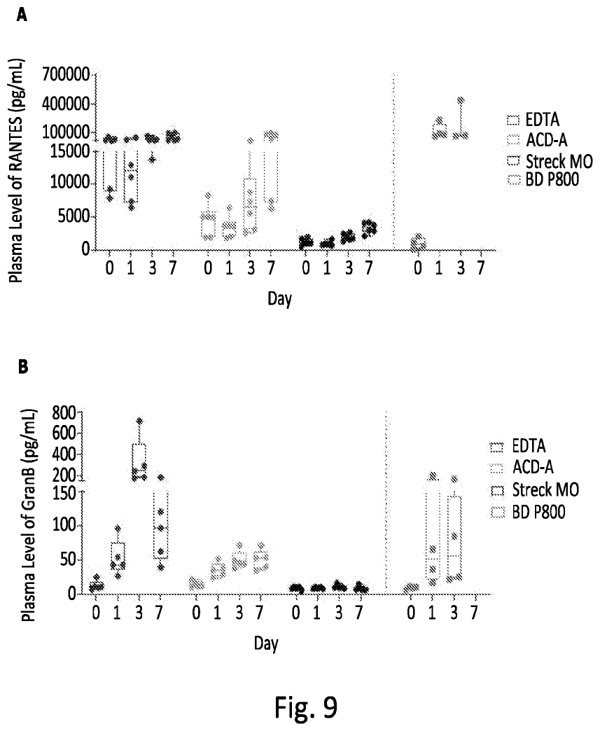

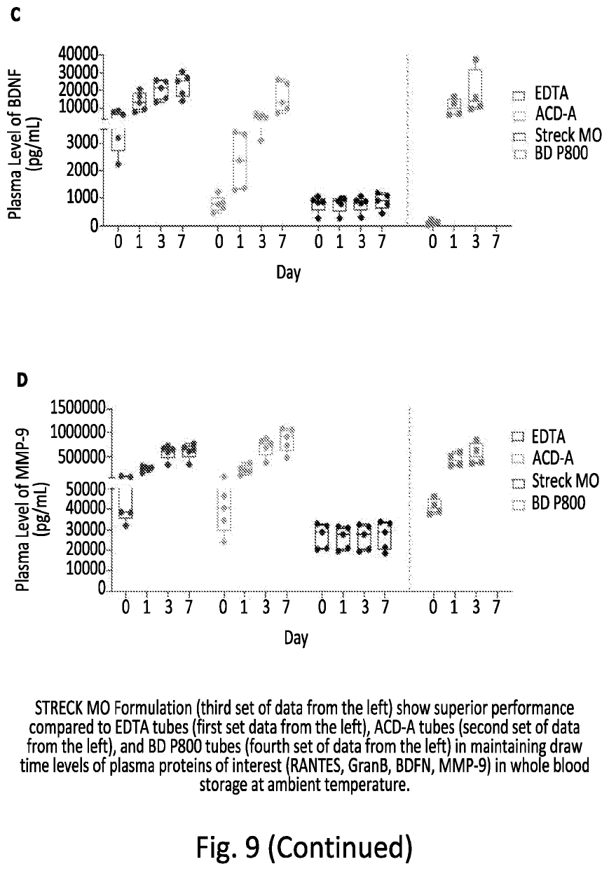

Capillary blood samples were collected from 3 donors. Per donor, two sets EDTA tube-paired Tasso+ devices and two sets of Streck-MO-filled tube-paired Tasso+ devices were used. As a reference, venous blood samples from the same 3 donors were collected using EDTA tubes and tubes prefilled with Streck MO formulation. Blood samples were stored at room temperature for up to 3 days. Three functionality assays were conducted, including measuring hemolysis (FIG. 8A) and testing the plasma level of MPO (FIG. 8B) and RANTES (FIG. 8C).

Capillary blood samples collected via Tasso+ had a higher level of in vitro Hemolysis compared to venous blood samples. Compared to EDTA-anticoagulated blood, Streck MO formulation minimized the hemolysis for up to 3 days in both venous blood and capillary blood. Streck MO formulation prevented granule release from activated platelets in both venous blood samples and capillary blood for up to 3 days during room temperature storage. In contrast, plasma levels of MPO and RANTES increased in EDTA-anticoagulated blood during room temperature storage.

### C. Spray-Coating Capillary Blood Collection Tube:

Capillary blood collection tubes spray coated with STRECK MO formulation have a minimum 6-month closed shelf-life at ambient temperature.

### Example 7—Stabilization Improvement of Streck MO Formulation for Protein Markers In Whole Blood Samples

Venous blood from 5 self-proclaimed healthy donors were drawn into EDTA tubes, ACD-A tubes, BD P800 blood collection tubes, and tubes pre-filled with Streck MO Formulation. Drawn whole blood samples were stored at room temperature for up to 7 days. Plasma protein biomarkers of interest were measured with immunoassay. Compared to blood samples collected in EDTA tubes, ACD-A tubes, or BD P800 tubes, Streck MO formulation maintained the draw time levels of plasma proteins of interest for up to 7 days of room temperature storage in whole blood. Whereas, in plasma samples obtained from BD P800 tubes, platelet activation was observed as early as day 1 of storage, as indicated by elevated plasma levels of RANTES (FIG. 5A). Furthermore, protein biomarkers such as Granzyme B (GranB, FIG. 5B), Brain-Derived Neurotrophic Factor (BDFN, FIG. 5C), and Matrix Metallopeptidase 9 (MMP-9, FIG. 5D) exhibited significant increases during whole blood stored in BD P800 tubes to granule content release upon in vitro platelet activation.

### Example 8—Functional Studies on Spray-Coated Microcollection Tubes

Blood was collected from healthy donors, each collected into 1×5 mL EDTA vacutainer tubes and virgin (no anticoagulant). Next, venous blood was aliquoted as quickly as possible into the spray-coated microtainers. The EDTA blood was aliquoted into virgin Greiner microtainers as negative controls. The blood with no anticoagulant was aliquoted into microcollection tubes spray coated with one of three formulations noted in Table 2 below, with two tubes per formulation. The sprayed microtainers were then mixed 15-20 times with the blood sample.

TABLE 2

| | Citric | Trisodium | | Imidazolidinyl | | | |
|---|---|---|---|---|---|---|---|
| | acid | citrate | Dextrose | Urea | α-cyclodextrin | Verapamil | Mexiletine |
| Formulation | (g/L) | (g/L) | (g/L) | (g/L) | (g/L) | (g/L) | (g/L) |
| Streck M2 | 25 to 35 | 125 to 135 | 140 to 150 | 140 to 160 | 25 to 35 | 0 | 5 to 10 |
| Streck MH1.5 | 18 to 23 | 92 to 105 | 105 to 115 | 140 to 160 | 18 to 23 | 6 to 8 | 0 |
| Streck MH | 12 to 17 | 60 to 70 | 68 to 78 | 140 to 160 | 10 to 17 | 3 to 5 | 0 |

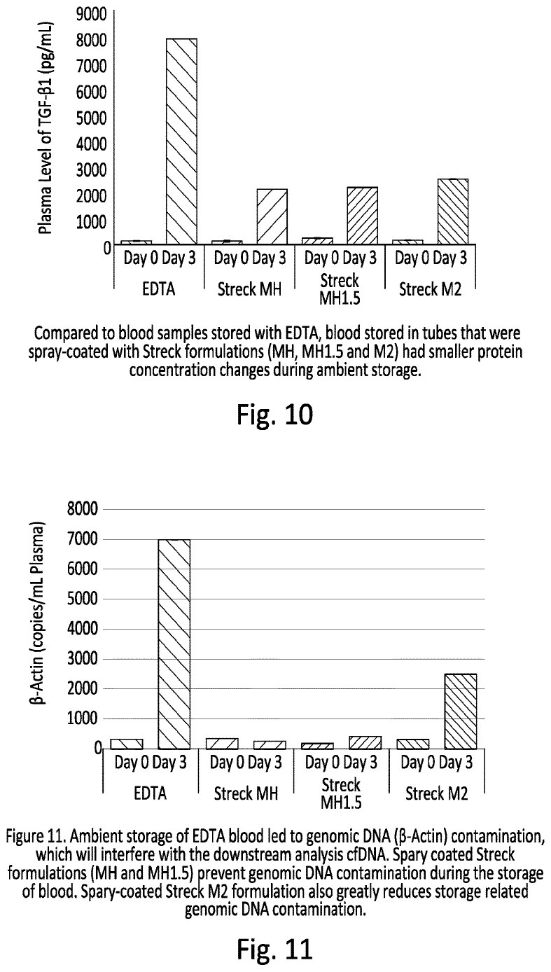

Half of the samples were analyzed at draw time and the remaining half were stored at room temperature for 3 days before analysis. Plasma protein concentration of TGF-β1 was analyzed with Ella (FIG. 10). Plasma β-Actin level was determined by ddPCR (FIG. 11).

As shown in FIG. 10, plasma TGF-β1 level in EDTA blood drastically increased after 3-day storage. In comparison, blood stored in spray-coated tubes had less increase after storage. This indicates Streck MH, MH1.5, and M2 formulations help to decrease storage induced changes of plasma protein concentration.

Elevated level of β-Actin DNA (ACTB) in plasma signals genomic DNA contamination. As shown in FIG. 11, the level of β-Actin in 3-day old EDTA plasma was more than 20-fold higher than that of draw time plasma. In contrast, the level of plasma β-Actin DNA in blood stored for 3 days with Streck MH formulation or Streck MH1.5 formulation is similar to that of draw-time blood. In addition, compared to EDTA blood, blood stored with Streck M2 formulation has much smaller changes in β-Actin level during storage.

### Example 8—Extended Shelf-Life of Streck MO and Streck MH Formulations

Blood collection tubes filled with preservative formulations (Streck MO or Streck MH) that were stored at 22° C. for up to 9 months. Freshly made tubes, 6 months old tubes, and 9 months old tubes were then used to collect venous blood from healthy donors. Blood specimens were stored at room temperature for up to 3 days.

Plasma levels of MPO and TGF-β1 were measured at draw time or at day 3 with Ella. Relative level of protein marker at day 3 was calculated by dividing the Day 3 protein concentration with Day 0 protein concentration. The result is listed in Table 3 below.

TABLE 3
Streck MO and Streck MH have at least 9 months of shelf-life. 6 months old and 9 months
old Streck formulations (MO and MH) behaved on par with the freshly made formulations
in maintaining draw-time levels of plasma proteins during ambient storage.

| | Relative MPO Level at Day 3 | | | Relative TGF-β1 Level at Day 3 | | |
|---|---|---|---|---|---|---|
| | (% Day 0) | | | (% Day 0) | | |
| Dating of the | | 6 months | 9 months | | 6 months | 9 months |
| Formulations | Fresh | old | old | Fresh | old | old |
| Streck MO | 100% | 100% | 100% | 100% | 100% | 100% |
| Streck MH | 100% | 100% | 100% | 100% | 94% | 100% |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

What is claimed is:

1. A composition comprising a citrate-based anticoagulant (AC), an aldehyde releaser (AR), a calcium channel blocker, and a sodium channel blocker.

2. The composition of claim 1, comprising AR and AC as an AC to AR ratio of about 4:1 to about 1:6.

3. The composition of claim 1, comprising AR and AC at an AC to AR ratio of about 2:1 to about 1:6.

4. The composition of any one of claims 1-3, comprising AR and AC at an AC to AR ratio of about 1:1.2.

5. The composition of any one of claims 1-4 wherein the citrate-based AC comprises acid citrate dextrose (ACD), citrate, citrate-theophylline-adenosine-dipyridamole (CTAD), citrate-pyridoxalphosphate-tris, citrate-dextrose-phosphate-adenine (CDPA), citrate-phosphate-dextrose-adenine (CPDA), or a combination thereof.

6. The composition of any one of claims 1-5, wherein the citrate-based AC is acid citrate dextrose (ACD).

7. The composition of any one of claims 1-6, comprising about 50 g/l to about 400 g/l AC.

8. The composition of any one of claims 1-7, wherein the AR is diazolidinyl urea, imidazolidinyl urea, 1,3,5-tris(hydroxyethyl)-s-triazine, oxazolidines, 1,3-bis(hydroxymethyl)-5,5-dimethylimidazolidine-2,4-dione, quaternium-15, DMDM hydantoin, 2-bromo-2-nitropropane-1,3-diol, 5-bromo-5-nitro-1,3-dioxane, tris(hydroxymethyl) nitromethane, hydroxymethylglycinate, polyquaternium, benzylhemiformal, sodium hydroxymethylglycinate, polynoxylin, (ethylenedioxy) dimethanol, tetramethylolacetylenediurea, urotropine, glutaraldehyde, N-methylol-chloracetamide, chlormethylacylamino methanol, hexamidine, 4-[2-(Morpholin-4-ylmethyl)-2-nitro-butyl]morpholine, 1,3-Dimethyl-4,5-dihydroxyethyleneurea, dimethyloldihydroxyethyleneurea, dimethylolethyleneurea, dimethylolpropyleneurea, dimethylol urea, ethylene urea, (Z)-3-(Bis(2-hydroxyethyl)amino)-2-(2-hydroxyethyl-(hydroxymethyl)amino) prop-2-en-1-ol, 1,3,5-Triethyl-1,3,5-triazinane, glyoxalmonourein, MDM hydantoin, 4,4′-dimethylenedimorpholine, Methylol urea, 3,4,4-trimethyloxazolidine, 2,2′,2″-(1,3,5-Triazine-2,4,6-triyl)tri(ethan-1-ol), 4,4-dimethyloxazolidine, 4,4′-(2-Ethyl-2-nitrotrimethylene)dimorpholine, 4-(2-Nitrobutyl) morpholine, phenylmethoxymethoxymethylbenzene, hydantoin, 3-(Hydroxymethyl)-5,5-dimethylimidazolidine-2,4-dione, methylal, N-Methylethanolamine, 5-hydroxymethoxymethyl-1-1aza-3,7-dioxabicyclo[3.3.0]octane, dithio-bis(male-imidoethane), ortho-phthalaldehyde, terephthalaldehyde, tris(hydroxymethyl)-nitromethane, 2-imidazolidinone (4,5-dihydroxy-1,3-bis(hydroxymethyl)-, methylated), 7a-Ethyldihydro-1H,3H,5H-oxazolo(3,4-c)oxazole, Bioban CS 1135; 2,4-imidazolidinedione, 3-(hydroxymethyl)-5,5-dimethyl, bis(hydroxymethyl) urea; 2-[(Hydroxymethyl)amino]ethanol, polyoxymethylene; 1,3-thiazolidane-4-carboxylic acid, Tris(N-hydroxyethyl) hexahydrotriazine, or a combination thereof.

9. The composition of any one of claims 1-8, wherein the AR comprises imidazolidinyl urea.

10. The composition of any one of claims 1-9, comprising about 100 g/L to about 1000 g/L AR.

11. The composition of any one of claims 1-10, wherein the calcium channel blocker is verapamil, diltiazem or a combination thereof.

12. The composition of claim 11, wherein the calcium channel blocker is verapamil.

13. The composition of any one of claims 1-12, comprising about 1 g/L to about 10 g/L verapamil.

14. The composition of any one of claims 1-13, wherein the sodium channel blocker is carbamazepine, benzocaine, bupivacaine, lidocaine, procaine, mepivacaine, mexiletine, or a combination thereof.

15. The composition of any one of claims 1-14, wherein the sodium channel blocker is mexiletine.

16. The composition of any one of claims 1-15, comprising about 0.4 g/L to about 45 g/L mexiletine.

17. The composition of any one of claims 1-16, further comprising a red blood cell (RBC) stabilizer.

18. The composition of claim 17, wherein the RBC stabilizer comprises a cyclodextrin.

19. The composition of claim 18, wherein the cyclodextrin is α-cyclodextrin, β-cyclodextrin or γ-cyclodextrin.

20. The composition of any one of claims 1-19, further comprising one or more of a P2Y12 receptor inhibitor, a COX-1 inhibitor, a GPlb inhibitor and an ATP release inhibitor.

21. The composition of any one of claims 1-20, further comprising one or more of cyclooxygenase inhibitors, ADP receptor inhibitors, Phosphodiesterase inhibitors, Protease-active receptor-1 antagonists, Glycoprotein IIB/IIIA inhibitors Adenosine reuptake inhibitors, peptide-like inhibitors, thromboxane inhibitors.

22. The composition of any one of claims 1-21, that comprises citric acid, trisodium citrate, imidazolidinyl urea, verapamil hydrochloride and mexiletine hydrochloride.

23. The composition of any one of claims 1-22, comprising 10 g/L to 40 g/L citric acid, 50 g/L to 150 g/L trisodium citrate, 65 g/L to 160 g/L dextrose, 140 g/L to 160 g/L imidazolidinyl urea, 1 g/L to 10 g/L verapamil and 1 to 10 g/L mexiletine.

24. The composition of any one of claims 1-22, comprising 10 g/L to 20 g/L citric acid, 50 g/L to 70 g/L trisodium citrate, 65 g/L to 85 g/L dextrose, 140 g/L to 160 g/L imidazolidinyl urea, 1 g/L to 10 g/L verapamil, and 1 g/L to 10 g/L mexiletine.

24. The composition of any one of claims 22-24, further comprising α-cyclodextrin.

26. The composition of any one of claims 1-25, that is formulated into a blood collection device.

27. The composition of claim 26, wherein the blood collection device is a blood collection tube (BCT).

28. The composition of claim 26, wherein the blood collection device is a micro collection tube or a capillary blood collection tube.

29. The composition of any one of claims 26-28, that is in liquid form within the blood collection device.

30. The composition of any one of claims 26-28, that is in lyophilized form within the blood collection device.

31. The composition of any one of claims 26-28, that is spray-coated onto the interior sides of the blood collection device.

32. A method of stabilizing a blood sample comprising contacting a blood sample with a composition according to any one of claims 1-31, wherein the composition stabilizes fractions within the blood sample.

33. The method of claim 32, further comprising isolating a cellular fraction of the blood sample.

34. The method of claim 33, wherein the cellular fraction is composed of rare blood cells and/or circulating nuclear cells.

35. The method of claim 34, wherein the rare blood cells are circulating tumor cells (CTCs), fetal circulating cells, rare white blood cells, or other rare circulating nuclear cells.

36. The method of claim 34, wherein the circulating nuclear cells comprise circulating white blood cells, such as Peripheral blood mononuclear cells and nucleated red blood cells.

37. The method of any one of claims 33-35, wherein the isolating step comprises separating rare blood cells in the sample from red blood cells, white blood cells, platelets, or a combination thereof.

38. The method of any one of claims 33-35, and 36, wherein the isolating step comprises separating circulating nuclear cells in the sample from rare blood cells, non-nucleated cells, or a combination thereof.

39. The method of any one of claims 33-35 and 37, wherein the isolating step comprises separating rare blood cells in the blood sample from plasma proteins.

40. The method of any one of claims 33-34, 36, and 38, wherein the isolating step comprises separating circulating nuclear cells in the blood sample from plasma proteins.

41. The method of any one of claims 33-39 comprising conducting proteomic (or peptidomic) analysis on the rare blood cells or white blood cells.

42. The method of any one of claims 33-41, further comprising the step of lysing cells of the cellular fraction.

43. The method of claim 42, further comprising isolating a plasma fraction of the blood sample.

44. The method of claim 43, further comprising isolating one or more of DNA, RNA and extracellular vesicles from the plasma fraction.

45. A composition comprising a citrate-based anticoagulant (AC), an aldehyde releaser (AR), and a sodium channel blocker.

46. The composition of claim 45, wherein the citrate-based AC comprises acid citrate dextrose (ACD), citrate, citrate-theophylline-adenosine-dipyridamole (CTAD), citrate-pyridoxalphosphate-tris, citrate-dextrose-phosphate-adenine (CDPA), citrate-phosphate-dextrose-adenine (CPDA), or a combination thereof.

47. The composition of claim 45 or claim 46, wherein the citrate-based AC is acid citrate dextrose (ACD).

48. The composition of any one of claims 45-47, comprising about 50 g/l to about 200 g/l AC.

49. The composition of any one of claims 45-47, comprising about 50 g/l to about 400 g/l AC.

50. The composition of any one of claims 45-49, wherein the AR is diazolidinyl urea, imidazolidinyl urea, 1,3,5-tris(hydroxyethyl)-s-triazine, oxazolidines, quaternium-15, DMDM hydantoin, 2-bromo-2-nitropropane-1,3-diol, 5-bromo-5-nitro-1,3-dioxane, tris(hydroxymethyl) nitromethane, hydroxymethylglycinate, polyquaternium, benzylhemiformal, sodium hydroxymethylglycinate, polynoxylin, (ethylenedioxy)dimethanol, tetramethylolacetylenediurea, urotropine, glutaraldehyde, N-methylol-chloracetamide, chlormethylacylamino methanol, hexamidine, 4-[2-(Morpholin-4-ylmethyl)-2-nitro-butyl]morpholine, 1,3-Dimethyl-4,5-dihydroxyethyleneurea, dimethyloldihydroxyethyleneurea, dimethylolethyleneurea, dimethylolpropyleneurea, dimethylol urea, ethylene urea, (Z)-3-(Bis(2-hydroxyethyl)amino)-2-(2-hydroxyethyl-(hydroxymethyl)amino) prop-2-en-1-ol, 1,3,5-Triethyl-1,3,5-triazinane, glyoxalmonourein, MDM hydantoin, 4,4′-dimethylenedimorpholine, Methylol urea, 3,4,4-trimethyloxazolidine, 2,2′,2″-(1,3,5-Triazine-2,4,6-triyl)tri(ethan-1-ol), 4,4-dimethyloxazolidine, 4,4′-(2-Ethyl-2-nitrotrimethylene)dimorpholine, 4-(2-Nitrobutyl) morpholine, phenylmethoxymethoxymethylbenzene, hydantoin, 3-(Hydroxymethyl)-5,5-dimethylimidazolidine-2,4-dione, methylal, N-Methylethanolamine, 5-hydroxymethoxymethyl-1-1aza-3,7-dioxabicyclo[3.3.0]octane, dithio-bis(male-imidoethane), ortho-phthalaldehyde, terephthalaldehyde, tris(hydroxymethyl)-nitromethane, 2-imidazolidinone (4,5-dihydroxy-1,3-bis(hydroxymethyl)-, methylated), 7a-Ethyldihydro-1H,3H,5H-oxazolo(3,4-c)oxazole, Bioban CS 1135; 2,4-imidazolidinedione, 3-(hydroxymethyl)-5,5-dimethyl, bis(hydroxymethyl) urea; 2-[(Hydroxymethyl)amino]ethanol, polyoxymethylene, 1,3-thiazolidane-4-carboxylic acid, Tris(N-hydroxyethyl) hexahydrotriazine, or a combination thereof.

51. The composition of any one of claims 45-50, wherein the AR comprises imidazolidinyl urea.

52. The composition of any one of claims 45-51, comprising about 100 g/L to about 1000 g/L AR.

53. The composition of any one of claims 45-52, wherein the sodium channel blocker is carbamazepine, benzocaine, bupivacaine, lidocaine, procaine, mepivacaine, mexiletine, or a combination thereof.

54. The composition of any one of claims 45-53, wherein the sodium channel blocker is mexiletine.

55. The composition of any one of claims 45-54, comprising about 0.4 g/L to about 45 g/L mexiletine.

56. The composition of any one of claims 45-55, further comprising a red blood cell (RBC) stabilizer.

57. The composition of claim 56, wherein the RBC stabilizer comprises a cyclodextrin.

58. The composition of claim 57, wherein the cyclodextrin is α-cyclodextrin, β-cyclodextrin or γ-cyclodextrin.

59. The composition of any one of claims 45-55, comprising 10 g/L to 40 g/L citric acid, 50 g/L to 150 g/L trisodium citrate, 65 g/L to 160 g/L dextrose, 140 g/L to 160 g/L imidazolidinyl urea, and 1 to 10 g/L mexiletine.

60. The composition of any one of claims 45-59, comprising about 15 g/L to about 40 g/L α-cyclodextrin.
